(19) United States
(12) Patent Application Publication
(10) Pub. No.: US 2024/0075254 A1
(43) Pub. Date: Mar. 7, 2024

(54) CATHETER DELIVERY DEVICES, SYSTEMS, AND METHODS

(71) Applicant: Piper Access, LLC, Salt Lake City, UT (US)

(72) Inventors: Jay Allen Muse, Salt Lake City, UT (US); Kevin Jerry Cook, Kaysville, UT (US)

(73) Assignee: Piper Access, LLC, Salt Lake City, UT (US)

(21) Appl. No.: 18/232,333

(22) Filed: Aug. 9, 2023

Related U.S. Application Data
(60) Provisional application No. 63/396,227, filed on Aug. 9, 2022.

(51) Int. Cl. A61M 25/06; A61M 25/00

(52) U.S. Cl. CPC A61M 25/0606; A61M 25/0023; A61M 25/0068; A61M 25/0097; A61M 25/065

(57) ABSTRACT

An apparatus can include a needle with a distal tip for insertion into a vessel of a patient, the needle defining an outer surface. The apparatus can include a catheter with a distal end and an inner surface that defines a lumen through which the needle extends. The apparatus can include a longitudinally extending stiffener positioned between the outer surface of the needle and the inner surface of the catheter. The stiffener can be movable to a distally advanced position relative to the needle to deploy the catheter and can be removable from the catheter along with the needle while in the distally advanced position. The stiffener can include a region of weakness at which the stiffener is predisposed to bend, which is closer to the distal tip of the needle than to the distal tip of the stiffener when the stiffener is in the distally advanced position.

### CROSS-REFERENCE TO RELATED APPLICATIONS

This application claims priority benefit under 35 U.S.C. § 119(e) of U.S. Provisional Patent Application No. 63/396,227, filed Aug. 9, 2022, titled CATHETER DELIVERY DEVICES, SYSTEMS, AND METHODS, the entire contents of which are hereby incorporated by reference herein.

### TECHNICAL FIELD

Certain embodiments described herein relate generally to catheters, and further embodiments relate more particularly to catheter delivery devices, systems, and methods.

### BACKGROUND

Many catheters are introduced into a patient via insertion needles. Some catheter systems include a catheter that is positioned over an insertion needle prior to introduction of the catheter into the patient. At least a distal tip of the needle can extend past a distal end of the catheter, and the distal end of the catheter may be tipped so as to have a smaller diameter than does a remainder of the catheter. The distal tip of the needle can be inserted into a vessel of the patient, and the catheter can follow through the opening thus created by the needle. Some systems exist for advancing the catheter over the needle and into the vessel. Known devices, systems, and methods, however, suffer from one or more drawbacks that can be resolved, remedied, ameliorated, or avoided by certain embodiments described herein.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of an embodiment of a catheter delivery system in a pre-use, retracted, undeployed, or pre-actuation operational state;

FIG. 2 is a cross-sectional view of the catheter delivery system of FIG. 1, taken along the view line 2-2 in FIG. 1, in the pre-use, retracted, undeployed, or pre-actuation operational state;

FIG. 3 is a perspective view of a lower end of an embodiment of an upper housing element compatible with the catheter delivery system;

FIG. 4 is an enlarged view of the upper housing element of FIG. 3 taken along the view line 4 in FIG. 3;

FIG. 5 is a perspective view of an embodiment of a lower housing element compatible with the catheter delivery system;

FIG. 6 is a perspective view of an embodiment of a catheter connection hub compatible with the catheter delivery system;

FIG. 7 is a perspective view of an embodiment of a catheter hub core compatible with the catheter delivery system;

FIG. 8 is a cross-sectional view of the catheter hub core of FIG. 7 and an embodiment of a catheter in a fixedly secured state;

FIG. 9 is a perspective view of an embodiment of an upper actuator compatible with the catheter delivery system;

FIG. 10 is a perspective view of an embodiment of a lower actuator or stiffener hub compatible with the catheter delivery system;

FIG. 11 is a cross-sectional view of the lower actuator of FIG. 10 and an embodiment of a stiffener in a fixedly secured state, which may be referred to collectively as a stiffener assembly;

FIG. 12 is a cross-sectional view of a distal portion of the catheter delivery system of FIG. 1 in the pre-use, retracted, undeployed, or pre-actuation state that is also depicted in FIGS. 1 and 2;

FIG. 13 is a cross-sectional view of a distal end of the catheter delivery system in the operational state depicted in FIG. 12, taken along the view line 13 in FIG. 12;

FIG. 14 is another cross-sectional view of the catheter delivery system of FIG. 1, similar to the view shown in FIG. 2 but depicting the catheter delivery system in a partially deployed or preliminarily actuated state;

FIG. 15 is another cross-sectional view of the catheter delivery system of FIG. 1, similar to the view shown in FIG. 2 but depicting the catheter delivery system in a nearly fully deployed or almost fully actuated state just prior to an embodiment of a catheter assembly transitioning to a coupled orientation;

FIG. 16 is another cross-sectional view of the catheter delivery system of FIG. 1, similar to the view shown in FIG. 2 but depicting the catheter delivery system in a fully deployed or fully actuated state in which the catheter assembly is in a coupled orientation, wherein the catheter delivery system includes a non-return feature that prevents retraction of a stiffener relative to a housing after full deployment is achieved;

FIG. 17 is another cross-sectional view of the catheter delivery system of FIG. 1, similar to the view shown in FIG. 2 but depicting the catheter delivery system in the fully deployed or fully actuated state and with the upper actuator having been retracted;

FIG. 18 is another cross-sectional view of the catheter delivery system of FIG. 1, similar to the view shown in FIG. 2 but depicting the catheter delivery system in the fully deployed or fully actuated state, with the upper actuator having been retracted, and with the catheter assembly having been decoupled from a handle or housing portion of the catheter delivery system; stated otherwise, FIG. 18 is a cross-sectional view of an insertion assembly portion of the catheter delivery system in a fully actuated state with the catheter assembly portion of the catheter delivery system having been removed therefrom;

FIG. 19 is a cross-sectional view of the catheter assembly in the coupled or assembled state after such has been decoupled from the insertion assembly portion of the catheter delivery system (e.g., shown in FIG. 18);

FIG. 20 is a side elevation view of a portion of an embodiment of a stiffener that is compatible with embodiments of the stiffener assembly of FIG. 11;

FIG. 21 is an enlarged side elevation view of an intermediately positioned portion of the stiffener of 20 taken along the view line 21 in FIG. 20;

FIG. 22 is a perspective view of the intermediately positioned portion of the stiffener of 21;

FIG. 23 is a cross-sectional view of a distal portion of the insertion assembly of FIG. 18 in the fully deployed state, after the insertion assembly has been removed from a catheter assembly portion of the catheter delivery system, with a stiffener thereof shown in an advanced state relative to an insertion needle thereof;

FIG. 24 is an enlarged cross-sectional view of a portion of an insertion assembly of FIG. 23 that corresponds with the region demarcated by the view line 24 in FIG. 23;

FIG. 25A is a side elevation view of a distal portion of a needle and an intermediate and distal portion of a stiffener in a shielding state, wherein the stiffener is shown in a substantially rectilinear state;

FIG. 25B is a side elevation view of the same portion of the needle and stiffener of FIG. 25A showing the stiffener having been bent at a region of weakness;

FIG. 26A is a side elevation view of a distal portion of a needle and an intermediate portion of a stiffener that is in a shielding state, wherein the stiffener is shown in a substantially rectilinear state; and

FIG. 26B is a side elevation view of the same portion of the needle and stiffener of FIG. 26A showing the stiffener having been bent at a region of weakness.

### DETAILED DESCRIPTION

The present disclosure relates generally to devices, systems, and methods for delivering catheters into the vasculature of patients. While specific examples of catheters are discussed with respect to the drawings, this discussion applies equally to additional types of catheters that may not specifically be shown or mentioned. For example, while some of the catheters depicted in the drawings and described in detail herein may be relatively short, some or all of the features described with respect to these shorter catheters may be advantageous in certain embodiments that have longer catheters, or stated otherwise, that are capable of deploying a catheter to a relatively greater depth within a vessel of a patient.

Certain known catheter delivery devices, systems, and methods involve over-the-needle catheters. Such systems can include a catheter and a needle that extends through a distal end of the catheter. In many cases, the catheter is attached to at least a distal end of the needle. In certain embodiments, a catheter delivery system can include a stiffener that can remain extended relative to the needle when the stiffener and the needle are removed from the catheter, and the stiffener can thereby act as a shield to prevent inadvertent contact with the distal tip of the needle. That is, the stiffener can function as a safety shield to prevent needle sticks. In some embodiments, the stiffener may be locked in an advanced position to ensure such shielding. Embodiments of over-the-needle catheter systems that include stiffeners to assist in the delivery of catheters into vasculature and/or to thereafter shield the needle are described in U.S. Pat. No. 11,065,419, titled CATHETER DELIVERY DEVICES, SYSTEMS, AND METHODS, which issued on Jul. 20, 2021 (the '419 patent). The entire contents of the '419 patent are hereby incorporated by reference herein.

In some embodiments, a stiffener may be formed as an elongated tube that encompasses a portion of the needle when the stiffener is in a retracted state. A distal tip of the stiffener can be positioned proximally relative to the needle tip when the stiffener is in the retracted state. The stiffener can be advanced distally relative to the needle to advance the catheter over and beyond the distal tip of the needle, such as into and/or through a blood vessel of the patient. The distal tip of the stiffener can remain positioned distally relative to the distal tip of the needle so as to sheath the needle while in this advanced or deployed state.

The stiffener can be locked in the advanced state. For example, in some instances, the stiffener may remain somewhat longitudinally movable relative to the needle after having been advanced, but a stop may prevent significant proximal retraction of the stiffener relative to the needle, such that the stiffener remains distally extended relative to the needle even when moved to its proximal-most orientation when in the somewhat moveable yet locked state. Stated otherwise, the lock may prevent significant proximal movement of the stiffener relative to the needle, but may not necessary prevent all movement of the stiffener relative to the needle, or stated yet another way, the locking mechanism may delimit proximal movement of the stiffener relative to the needle once engaged, activated, or otherwise in use. The lock may yield a minimum sheathing distance to which the stiffener extends distally beyond the distal tip of the needle. Certain embodiments of such locking mechanisms are described in the '419 patent. In other instances, as also discussed in the '419 patent, when the stiffener is in a locked state relative to the needle, the stiffener may instead be secured in a fixed longitudinal orientation relative to the needle, such that relative longitudinal movement between the stiffener and the needle is not possible when the stiffener is in the locked position. In either case, when the stiffener is in a locked state, a significant length of the stiffener can be retained in a position that extends past the distal tip of the needle.

When the stiffener and the needle are removed from the catheter, particularly when the stiffener is retained in the locked state, the stiffener can shield a distal tip of the needle from contact. When the stiffener is in a substantially rectilinear configuration, the stiffener can extend distally beyond the distal tip of the needle by a substantial distance to prevent a user from inadvertent contact with the needle tip.

In some embodiments, the stiffener may be formed of a material that may be capable of lateral deflection. For example, in various embodiments, the stiffener may be formed of a polymeric material, such as PEEK. The greater the length of the stiffener that extends past the distal tip of the needle, the more prone the stiffener may be to bending. In some embodiments, the extended length of the stiffener may bend such that an internal surface of the sidewall of the stiffener comes into close proximity to, up to and including contact with, the needle tip. In some instances, the thickness and/or hardness of the sidewall may be such that the needle tip can pierce through the sidewall when or after such bending occurs.

For example, in some instances, the bending may be relatively likely to or may naturally occur at or near a pivot point of the needle tip. This may result from the extended length of the stiffener being unsupported at its interior, while the portion of the stiffener within which the needle resides is relatively stiffer as it is supported by the presence of the relatively stiff needle therein, particularly where there may be little space between the outer surface of the needle and the inner surface of the stiffener in some embodiments. The possibility of the needle tip being near or in direct contact with a folded-over portion of the stiffener, such that the needle tip may advance directly through the sidewall of the stiffener if force is applied externally on the sidewall, may reduce the effectiveness of the stiffener as a shielding element. That is, the needle tip might, in some instances, be capable of piercing through the sidewall and administering inadvertent needle sticks, which could be an undesirable hazard.

Certain embodiments described hereafter can be particularly well suited to ameliorate or eliminate one or more of the foregoing potential drawbacks. The embodiments may also or alternatively provide one or more additional or other advantages, as will be apparent from at least the written description.

For example, in certain embodiments, the stiffener can include a region of weakness at which the stiffener is predisposed to bend. This region of weakness can be a preferred bending point for the stiffener, such that the stiffener is more likely to bend at the region of weakness than at a position at or immediately adjacent to the needle tip. The region of weakness can be distanced from the distal tip of the needle by a predetermined amount, thereby yielding a shielding length of the stiffener that remains in a substantially rectilinear state and is distal to the needle tip so as to shield the needle tip when the stiffener is bent at the region of weakness. The shielding portion of the stiffener that is positioned longitudinally between the needle tip and the region of weakness can be sufficiently long to shield the needle tip, such as by preventing inadvertent contact therewith and/or by preventing the needle tip from puncturing through the sidewall of the folded-over portion of the stiffener, while also being sufficiently short to inhibit bending or buckling at the selfsame shielding portion. For example, this shielding portion of the stiffener may be sufficiently short to have a relatively high resistance to lateral bending and a relatively high resistance to longitudinal buckling. With the stiffener being predisposed to bend at a specific position, a distal end of the stiffener may strategically or sacrificially bend laterally in the event of inadvertent contact therewith. With the overall length of the stiffener that extends past the distal tip of the needle thus reduced, the shielding length of stiffener that remains unbent—that is, the shielding length of the stiffener that extends between the region of weakness and the distal tip of the needle—can provide robust and reliable shielding or protection to the distal tip of the needle, as discussed further below.

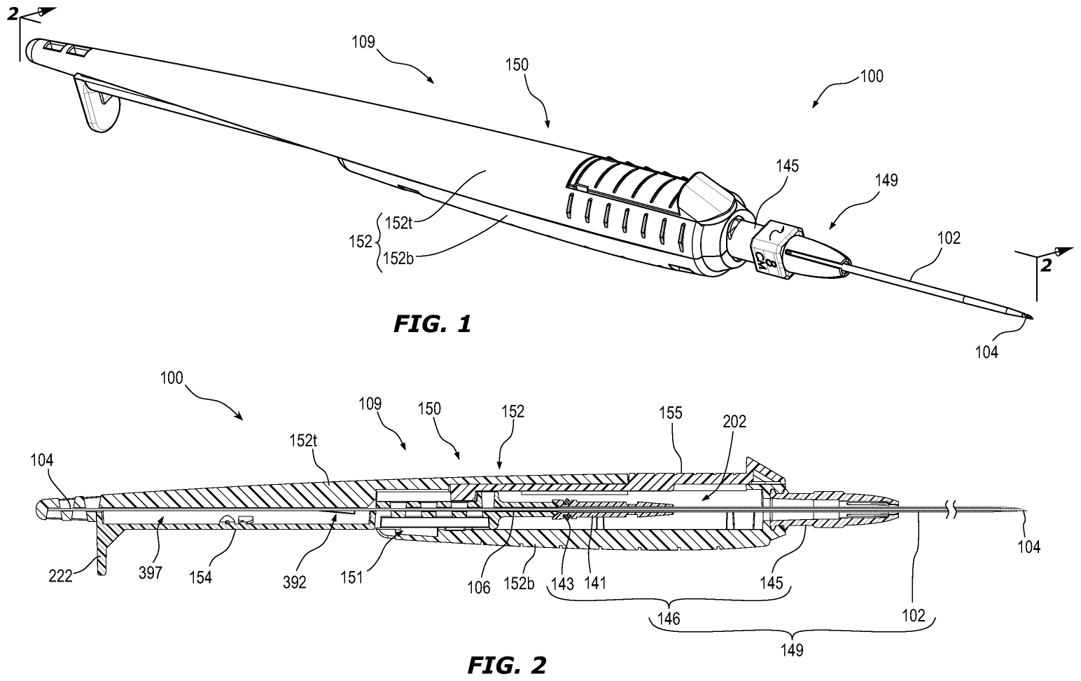

FIGS. 1 and 2 depict an embodiment of a catheter delivery system 100. In certain embodiments, certain components of the catheter delivery system 100 can resemble clearly analogous components of certain embodiments described in the '419 patent. Stated otherwise, the system 100 can include components that resemble certain components of systems described in the '419 patent (such as, e.g., components of the systems 800, 2000, 2100, and/or 2700) in certain respects. Accordingly, like features may be designated with like reference numerals, with the leading digits altered, where appropriate. Relevant disclosures set forth in the '419 patent regarding similarly identified features are incorporated hereby with respect to the system 100.

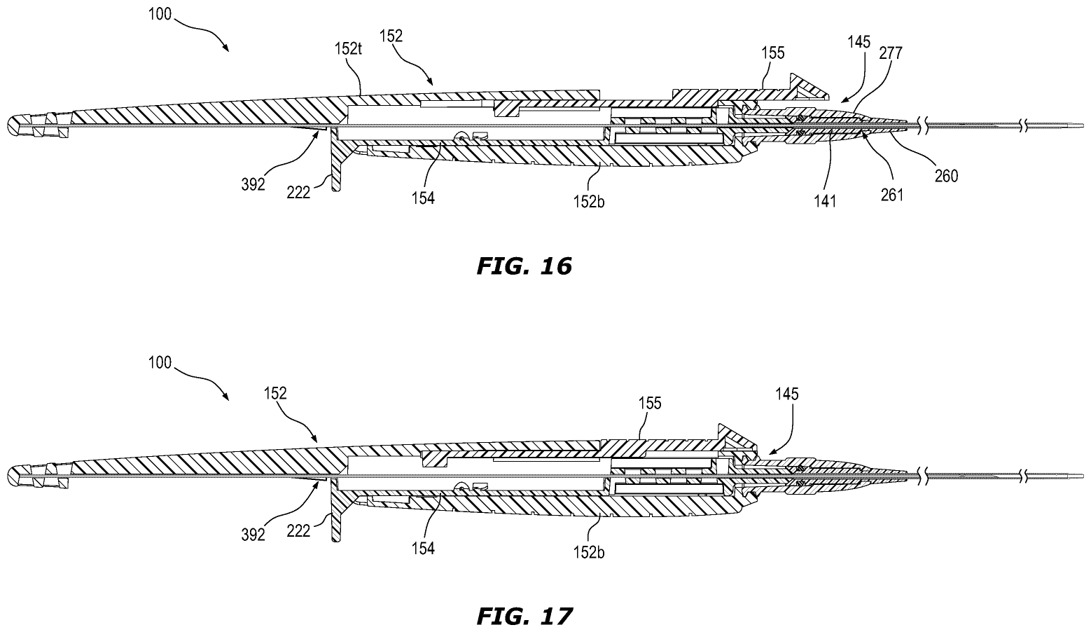

In the illustrated embodiment, the system 100 includes an insertion assembly 109 that is selectively coupled with a catheter assembly 149. As further discussed below, the system 100 is in a pre-use, retracted, undeployed, or pre-actuation operational state. The insertion assembly 109 is configured to deploy a catheter 102 to a desired depth within a vessel of a patient by advancing the catheter 102 over an insertion needle 104. In so doing, the insertion assembly 109 transitions the catheter assembly 149 from a disassembled state to an assembled state, as further discussed below. After deployment of the catheter 102 and after transition of the catheter assembly 149 to its assembled state, the insertion assembly 109 can be detached from the fully assembled catheter assembly 149 and withdrawn therefrom, thus leaving the catheter assembly 149 in place within the vasculature of the patient. Deployment of the catheter 102 and transition of the catheter assembly 149 to the assembled state are events of which at least a portion may occur simultaneously. For example, assembly of a hub portion of the catheter assembly 149 may occur during a final phase of deployment of the catheter 102, as discussed further below (e.g., with respect to FIGS. 15 and 16).

The catheter assembly 149 can include the catheter 102, a catheter hub core 141, a seal member 143, and a catheter connection hub 145. The catheter hub core 141 is secured to the catheter 102 (e.g., overmolded over a proximal end of the catheter 102). The seal member 143 can be coupled with the catheter hub core 141 in any suitable manner, such as by being positioned within a groove defined by the catheter hub core 141, as shown in FIGS. 2, 7 and 8. In the illustrated disassembled state of the catheter assembly 149, which can correspond to the undeployed state of the system 100 (shown in FIGS. 1 and 2), the catheter hub core 141 is spaced from and positioned rearward of (proximal to) the catheter connection hub 145, and the catheter 102 extends through an entirety the catheter connection hub 145. The catheter hub core 141, the seal member 143, and the catheter connection hub 145 can be assembled together to form a catheter hub 146, as shown in FIG. 19. Stated otherwise, FIG. 19 depicts the catheter assembly 149 in a fully assembled state. In this state, an assembled catheter hub 146 includes the catheter connection hub 145 fixedly secured to the catheter hub core 141. The seal member 143 forms a fluid-tight seal with the catheter connection hub 145.

With reference again to FIGS. 1 and 2, in the illustrated embodiment, the insertion assembly 109 includes a handle 150, which can comprise a housing 152. The housing 152 can be shaped to have an ergonomic contour that can be readily gripped by a hand of a user. The illustrated housing 152 includes a top or upper housing element 152t and a bottom or lower housing element 152b. The housing elements 152t, 152b can cooperate to define an opening, port, or channel 151 through which a stiffener hub 154 extends. The channel 151 is positioned at a mid or intermediate region of the housing 152.

In the pre-use, pre-deployment, initial, or as-packaged state depicted in FIGS. 1 and 2, the housing 152 is connected at a distal end thereof with the catheter connection hub 145. The catheter connection hub 145 can be selectively releasable from the housing 152. For example, in the illustrated embodiment, a distal end of the housing 152 can include internal threading 212, and a proximal end of the catheter connection hub 145 can include external threading 275 that is complementary to and selectively attachable to the threading of the housing via rotational engagement. (See, e.g., FIGS. 2, 3, 5, and 6.) Any other suitable connection interface is contemplated.

The insertion assembly 109 can further include a stiffener 106, which may also or alternatively be referred to as, or may have an alternate form that comprises at least one component that may be referred to as, a support, column, reinforcement, frame, scaffold, prop, strut, brace, spine, rod, tube, and/or cannula. For example, in the illustrated embodiment, the stiffener 106 may also be referred to as a sheathing cannula, a cannular stiffener, etc. In the illustrated embodiment, the stiffener 106 is formed of an elongated tube that is positioned between an outer surface of the insertion needle 104 and an inner surface of the catheter 102 when the system 100 is in the undeployed configuration (such as depicted in FIGS. 1 and 2). This nested or telescoped arrangement in the undeployed state is also depicted in FIG. 13.

The stiffener 106 may be flexible in the transverse dimension (e.g., in directions orthogonal to a longitudinal axis of the tube), yet may be substantially rigid or stiff in the axial direction to counteract axial forces (i.e., longitudinally directed force) applied thereto by the distal portion of the catheter 102 during insertion of the system 100 through the vessel wall and during advancement of the system 100 through the lumen of the vessel.

With reference to FIG. 2, the insertion needle 104 is positioned within the stiffener 106 and extends through an entirety of the catheter 102 (e.g., extends distally past a forward end of the catheter 102 and extends proximally past a rearward end of the catheter 102), through an entirety of the stiffener 106, and through most of the housing 152. In particular, the needle 104 extends through a distal end of the housing 152 and a proximal end of the needle 104 is attached, internally, to a proximal end of the upper housing element 152t.

With reference to FIGS. 2 and 11, the illustrated stiffener 106 is secured at a proximal end thereof to the stiffener hub 154, which is movable within and relative to the housing 152. In the illustrated embodiment, the stiffener 106 is formed as a tubular element that encompasses a portion of the needle 104. The stiffener 106 is movable relative to (e.g., over, along an exterior of) the needle 104.

The stiffener hub 154 can include an actuation element 222 may protrude away from the housing 152 to be engageable by the hand (e.g., one or more fingers) of a user. For example, the user may press distally on the actuation element 222 to thereby move the stiffener hub 154 and the attached stiffener 106 in a distal direction relative to the needle 104 and housing 152. The stiffener hub 154 may generally be referred to as an actuator, such as in the illustrated embodiment in which the stiffener hub 154 is rigidly fixed to (e.g., integrally formed with) the actuation element 222 such that the stiffener hub 154 and the actuation element 222 move in unison as a single body. Alternatively, the stiffener hub 154 may be said to be coupled to (e.g., integrally formed with or otherwise) an actuator. Thus, in the illustrated embodiment, a body portion of the stiffener hub 154 may be said to be attached to the actuation element 222. The actuation element 222 may also or alternatively be referred to as an actuator, a deployment actuator, an advancement actuator, a primary actuator, a first actuator, a direct stiffener hub actuator, a lower actuator, a rear actuator, etc. Moreover, in many instances, reference to the actuator 222 may more generally be understood as a reference to the stiffener hub 154 in its entirety.

With continued reference to FIG. 2, the insertion assembly 109 can further include an initiation actuator 155, which is described further below. The initiation actuator 155 may also be referred to as an insertion actuator, a stabilization actuator, a supplemental actuator, an optional actuator, a second actuator, an indirect stiffener hub actuator, an upper actuator, a forward actuator, etc. With respect to the designation of the actuators 222, 155 as lower or upper actuators, respectively, it should be understood that these terms refer to the positions depicted in the views shown in FIGS. 1 and 2, and are not limiting with respect to other arrangements. That is, the terms “upper” and “lower” are used illustratively herein for convenience, it being understood that these terms are readily substituted with other suitable appellations for the actuators 222, 155. For example, in other embodiments, the actuators 222, 155 are reversed, such that the actuator 222 is accessible at an upper end of the housing 152 and the actuator 155 is accessible at the lower end of the housing 152. In still other embodiments, the positions of the actuators 222, 155 may be fully altered, such as by being at lateral positions. For example, rather than being at opposing or opposite upper and lower sides of the housing 152, the actuators 222, 155 can be positioned at other opposing sides (e.g., left and right sides) of the housing 152.

The initiation actuator 155 can selectively couple with the stiffener hub 154 to move the stiffener hub 154 forward by an initial amount, as further discussed below. In the illustrated embodiment, when the system 100 is in the pre-use or pre-deployment configuration, the initiation actuator 155 can be adjacent to or in coupling contact with the stiffener hub 154 (see FIG. 2) such that forward or distal movement of the actuator 155 effects immediate (or nearly immediate, such as where the initiation actuator 155 moves a short distance prior to engaging the stiffener hub 154), simultaneous or concurrent forward movement of the stiffener hub 154 (and, thereby, forward movement of the stiffener 106 and catheter 102).

In some instances, the system 100 can be fully deployed using only the lower actuator 222. In other instances, the system 100 can be deployed in two separate phases: first, by advancing the upper actuator 155 to insert the catheter 102 to a first depth within the vessel of a patient; and second, by advancing the lower actuator 222 to further advance the catheter 102 to a second depth within the vessel that is greater than the first depth. Embodiments of the latter deployment technique are discussed below with respect to FIGS. 2 and 14-19.

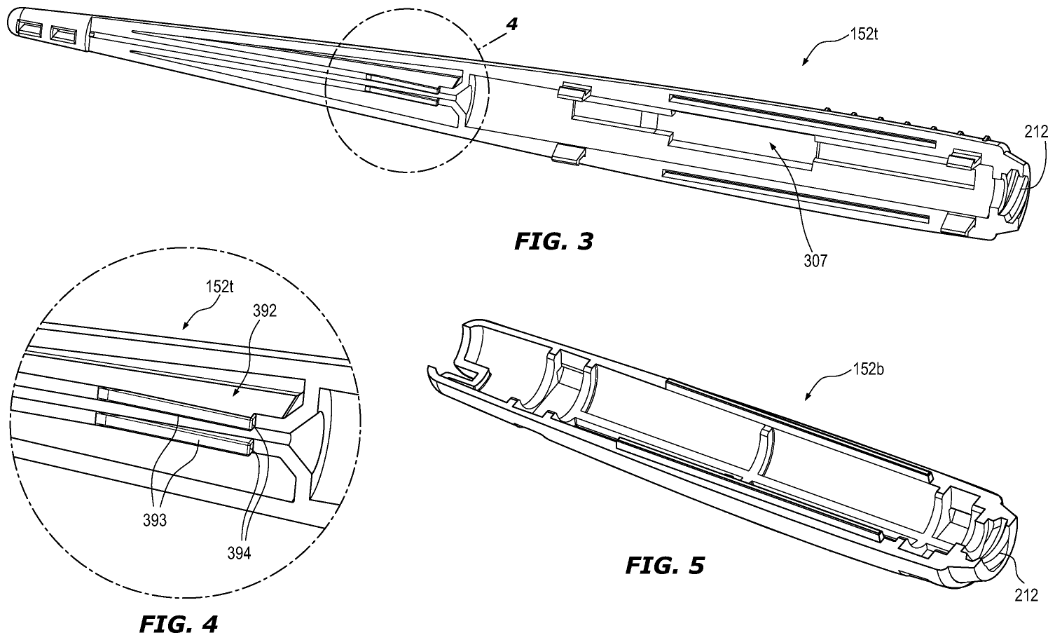

With reference to FIGS. 3-5, in some embodiments, one or more of the upper and lower housing elements 152t, 152b, the upper actuator 155 and/or the stiffener hub 154 are formed of a polycarbonate (PC) and acrylonitrile butadiene styrene (ABS) blend (i.e., PC/ABS). In other embodiments, one or more of the aforementioned components are formed of polycarbonate, acetal, etc. Any other suitable material and/or combinations thereof is/are contemplated.

The upper and lower housing elements 152t, 152b can be joined together in any suitable fashion. The upper actuator 155 and the stiffener hub 154 can be coupled with the housing 152 in any suitable manner so as to be moveable (e.g., translatable) relative thereto.

With reference to FIG. 4, the upper housing element 152t can define a stop 392, which can inhibit or prevent proximal movement of the stiffener hub 154, and thus proximal movement of the attached stiffener 106, after the stiffener hub 154 and stiffener 106 have been distally advanced by a sufficient amount relative to the needle 104, as discussed further below. The illustrated stop 392 includes a pair of ramps 393, each of which includes an abutment or engagement surface 394 at a distal end thereof. The ramps 393 of the stop 392 can project downwardly.

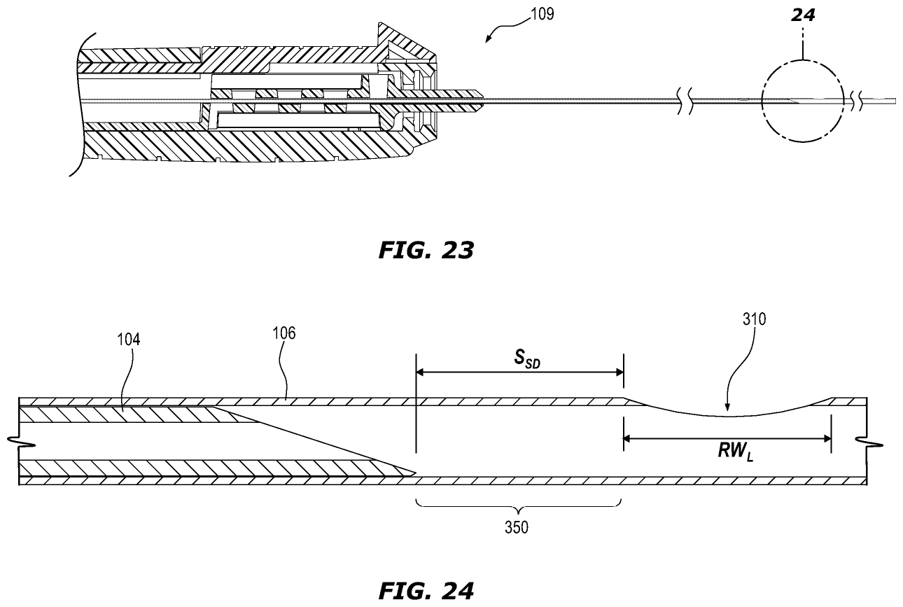

The stop 392 may be referred to as a non-return, locking, shielding, or safety feature. That is, the non-return feature prevents retraction of the stiffener 106 relative to the housing 152 after full deployment of the system 100. As further discussed below, once a proximal end of the stiffener hub 154 is advanced distally past the engagement surfaces 394 of the ramps 393, the engagement surfaces 394 interface with a proximal surface of the stiffener hub 154 to prevent the stiffener hub 154 from moving proximally relative to the housing 152. The stiffener hub 154 thus cannot be retracted relative to the housing 152. Thus, the stiffener 106 to which the stiffener hub 154 is attached is maintained in a fixed, shielding position over the needle tip (e.g., in a shielding position such as that depicted in FIG. 23).

In the illustrated embodiment, the stiffener 106 is maintained in the fully deployed position, and thus extends a maximum length past the needle tip. In other embodiments, the non-return feature may permit some amount of proximal movement of the stiffener hub and stiffener relative to the housing after deployment, but prevent full retraction of the stiffener from over the needle. That is, the system can maintain at least some length of the stiffener past the distal end of the needle in an amount sufficient to shield the needle tip from inadvertent contact. Stated otherwise, the system can retain the stiffener in at least a partially deployed state.

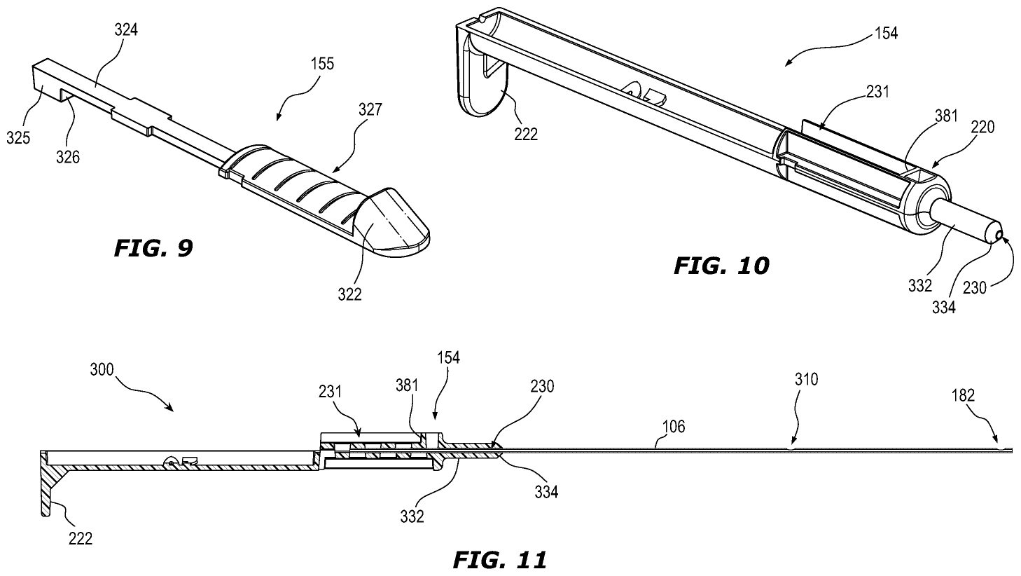

With reference to FIG. 9, the upper actuator 155 can include an actuation region 327 engageable by a user to advance the upper actuator 155 distally. In further embodiments, the actuation region 327 may include friction enhancing features, which may facilitate distal and/or proximal movement of the upper actuator 155. In some embodiments, a catch 322 can extend upwardly at a distal end of the actuation region 327.

The upper actuator 155 can include a longitudinal stem 324 that connects the actuation region 327 with an engagement protrusion 325. The stem 324 may also be referred to as a guide or slider. The engagement protrusion 325 that extends from the stem 324 can be configured to engage the stiffener hub 154 within the housing 152, as discussed further below. For example, in the illustrated embodiment, the engagement protrusion 325 includes an engagement face 326 that interferes with a surface of the stiffener hub 154 when the upper actuator 155 is advanced distally. The illustrated engagement face 326 is a substantially planar face at a distal end of the engagement protrusion 325 which, when positioned within the assembled system 100, extends substantially transverse or orthogonal to the longitudinal axis of the system 100.

The longitudinal stem 324 can be sized (e.g., can define a width) to fit within a track 307 of the upper housing element 152t (FIG. 3). As the upper actuator 155 is advanced or retracted along the track 307, the longitudinal stem 2324 can slide or otherwise translate within the track 307. The track 307 can constrain movement of the upper actuator 155, and in particular, can constrain movement of the longitudinal stem 324. For example, the track 307 can inhibit or prevent lateral movement of the longitudinal stem 324.

With reference to FIGS. 10 and 11, the stiffener hub 154 includes a body 220 that can define an internal channel 230 for coupling with the stiffener 106. The body 220 can define a track, guide path, or channel 231 at an upper end thereof through which the engagement protrusion 325 of the upper actuator 155 (FIG. 9) can pass. The engagement protrusion 325 can be sized to translate within the channel 231 in an uninhibited manner. The engagement face 326 of the engagement protrusion 325 (FIG. 9) can interface with (e.g., interfere with) an engagement face 381 at a distal end of the channel 231 to effect forward movement of the stiffener hub 154. For example, in the illustrated embodiment, the engagement face 381 defines a substantially planar surface that extends substantially orthogonally relative to a longitudinal axis of the assembled system 100. The opposing engagement faces 326, 381 of the channel 321 of the stiffener hub 154 and of the engagement protrusion 325 of the upper actuator 155, respectively, thus can interfere with each other, or selectively engage with each other, when the upper actuator 155 is advanced distally. The engagement faces 326, 381 can disengage from each other when the upper actuator 155 is retracted proximally.

The stiffener hub 154 can further include a protrusion 332 that extends distally from the body 220. The protrusion 332 can include a tapered tip 334 at a distal end thereof that fits within a tapered mouth 264 of the catheter hub core 141 (see FIG. 8). The tapered tip 334 of the stiffener hub 154 can directly engage the catheter hub core 141, and may function as (and be referred to as) an engagement member.

The body 220 of the stiffener hub 154 can be configured to readily pass through an internal chamber 202 defined the housing 152 (see FIG. 2). The chamber 202 may be said to constrain movement of the body 220 therein, such as to ensure a substantially linear path of movement that is substantially aligned with the longitudinal axis of the system 100.

With reference to FIGS. 7 and 8, the catheter hub core 141 can include a tapered distal end 260 and an engagement recess 261. In various embodiments, the catheter hub cored 141 can be formed of polyurethane, such as, for example, Isoplast®, available from Lubrizol, of Wickliffe, Ohio. Another illustrative example of a suitable material includes polycarbonate. Any other suitable material is contemplated.

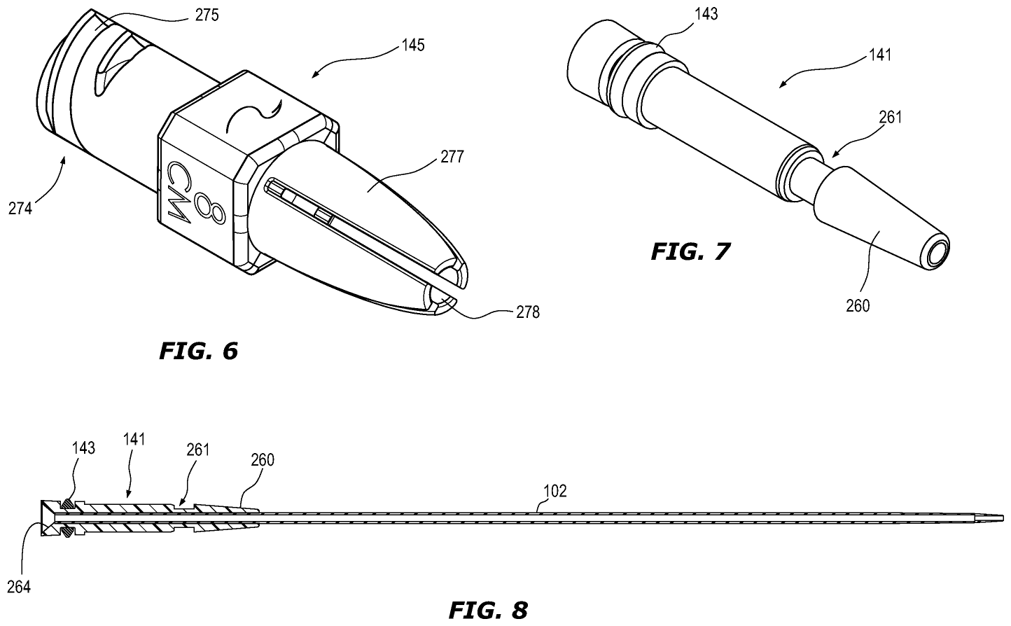

With reference to FIG. 6, the catheter connection hub 145 can include a connection interface 274 configured to selectively couple the catheter connection hub 145 to the housing 152 and selectively decouple the catheter connection hub 145 from the housing 152. For example, as previously mentioned, in the illustrated embodiment, the connection interface 274 comprises external threading 275. In other embodiments, one or more external lugs or protrusions that can suitably couple with the threading 212 of the housing 142 may be used in place of the threading 275. Any suitable connection interface 274 is contemplated.

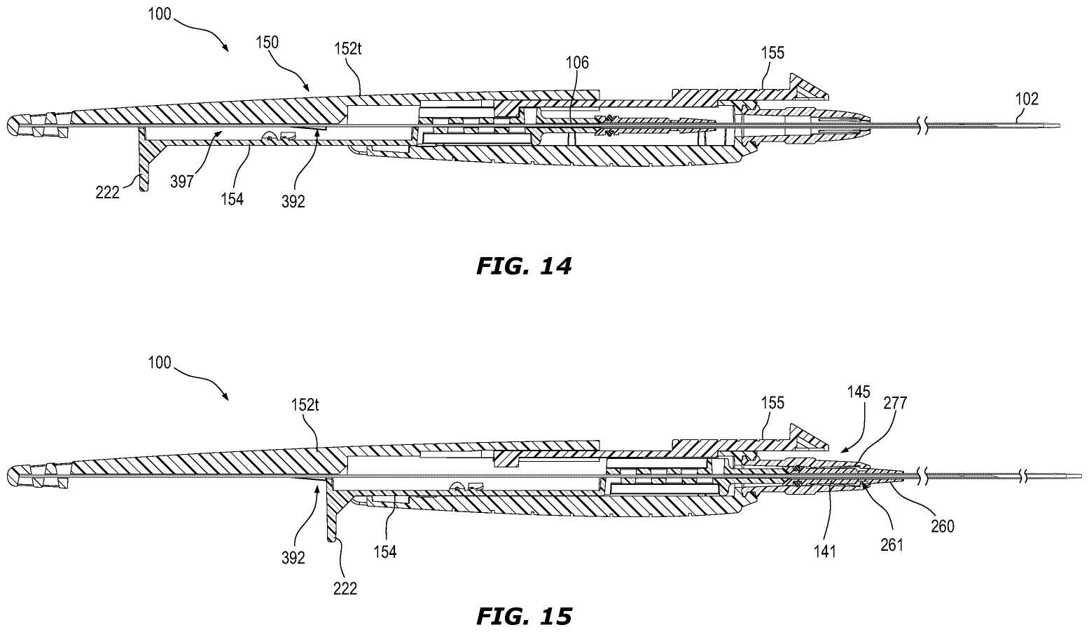

In some embodiments, the catheter connection hub 145 includes a plurality of engagement arms 277 at a distal end thereof. The engagement arms 277 can be configured to automatically couple the catheter connection hub 145 to the catheter hub core 141 during deployment of the catheter 102. In the illustrated embodiment, internally directed protrusions 278 at the distal ends of the respective engagement arms 277 interface with the tapered distal end 260 of the catheter hub core 141 as the catheter hub core 141 is advanced distally. The arms 277 deflect outwardly until the catheter hub core 141 has been advanced sufficiently to permit the engagement arms 277 to automatically resiliently deflect inwardly such that the internally directed protrusions 278 to seat within the engagement recess 261 of the catheter hub core 141 and thereby hold the catheter connection hub 145 in secure engagement with the catheter hub core 141. This process is depicted, e.g., in FIGS. 14-16.

In various embodiments, the catheter connection hub 145 is formed of polyurethane, such as, for example, Isoplast®, available from Lubrizol. Another illustrative example of a suitable material includes polycarbonate. Any suitable material is contemplated.

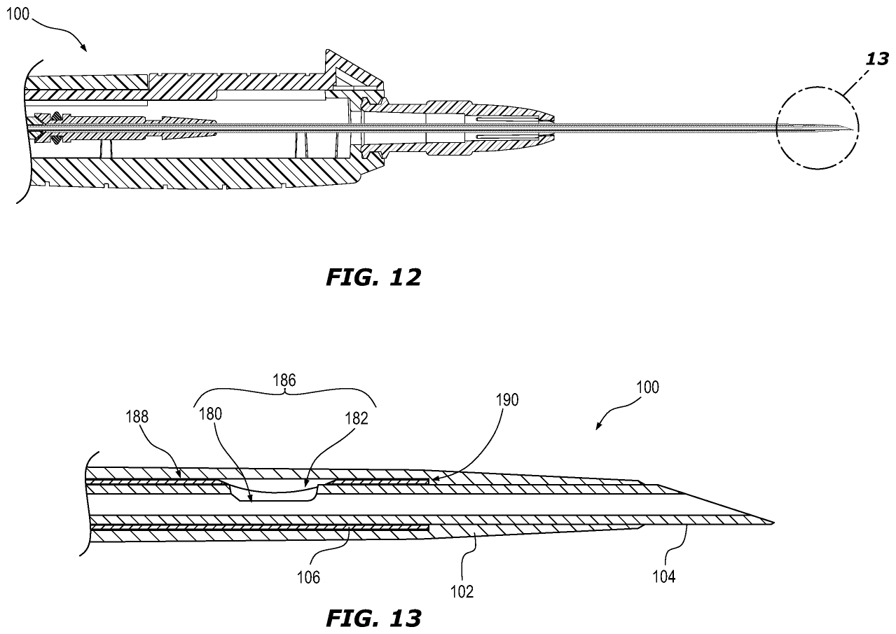

FIGS. 2 and 12-19 depict various stages of operation of the system 100. Stated otherwise, FIGS. 2 and 12-19 depict various stages or steps of illustrative methods, such as methods of using the system 100. Accordingly, the following discussion of these drawings disclose both operational details of embodiments of the system 100, as well as illustrative methods, including methods that specifically employ embodiments of the system 100.

FIGS. 2, 12, and 13 depict the system 100 in an undeployed, pre-use, as-packaged, or initial state. For example, the system 100 is shown in a state in which the system 100 may be sterilized, packaged, delivered to a user, and/or removed from packaging by the user. Stated otherwise, the user may, in some instances remove the system 100 from packaging in substantially the illustrated configuration.

In some embodiments, the system 100 includes a cap (not shown), which can cover the distal tip of the needle 104 to prevent inadvertent sticks prior to intended use. Any suitable mechanisms may also be employed to maintain the upper actuator 155 and the lower actuator 222 in their respective retracted states. For example, in some embodiments, the cap and/or a separate spacer or stop element (not shown) can be configured to maintain the upper actuator 155 in the fully retracted or undeployed orientation. Further, in some embodiments, a separate cap, spacer, or stop element and/or packaging for the system 100 can prevent actuation of the lower actuator, such as during transport.

The general arrangement of and relationships between the catheter 102, the needle 104, and the stiffener 106 have previously been described. As shown in FIG. 13, in the initial operational state, a distal end of the needle 104 can extend past a distal end of the catheter 102. The distal end of the catheter 102 can include a catching region 190 that can interface with a distal tip of the stiffener 106. As described further below, during deployment, the stiffener 106 can be advanced distally relative to the needle 104, which can press on the catching region 190 of the catheter 102. The stiffener 106 can serve to push the distal end of the catheter 102 off of and away from the distal end of the needle 104, and can assist in advancing the catheter 102 distally relative to the needle 104. For example, proximal portions of the catheter 102 may be seen as being pulled into the vasculature via the distal tip of the stiffener 106 as it remains in engagement with and pushes distally against the catching region 190.

As previously mentioned, and as shown in FIGS. 2, 12, and 13, in the initial or retracted state, the needle 104 can extend through an entirety of an interior of the stiffener 106. The proximal end of the needle 104 is fixedly secured to the housing 152. At least a distal portion of the stiffener 106 can be positioned within a lumen defined by the catheter 102. Stated otherwise, at least a portion of the stiffener 106 can be positioned between an inner surface defined by the catheter 102 and an outer surface defined by the needle 104. The catheter 102, stiffener 106, and needle 104 can be concentrically oriented, share a common longitudinal axis, and/or stated otherwise, can be in a nested configuration, as shown. The stiffener 106 can be translatable relative to the needle 104 and, at least initially, is translatable in a distal direction.

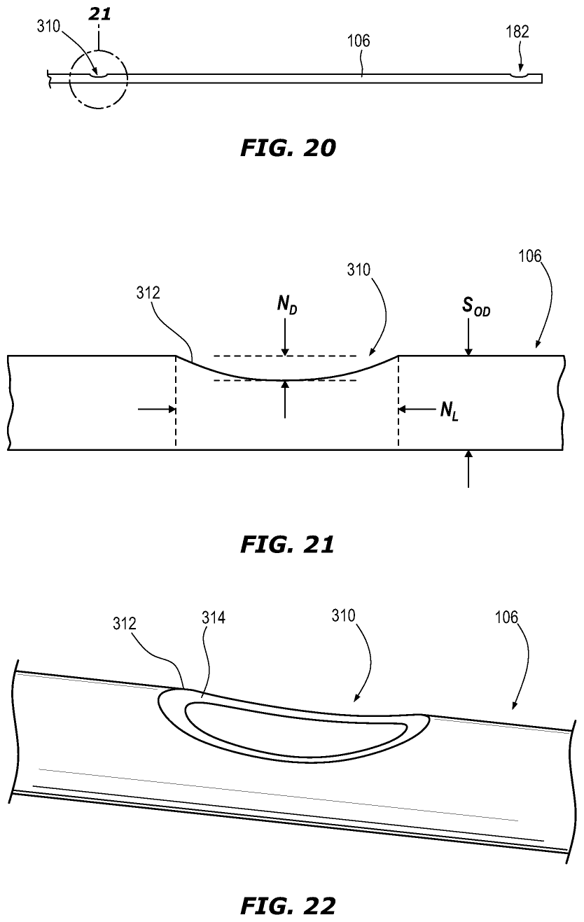

With reference to FIG. 13, when the system 100 is in the undeployed state, a port 180 through the sidewall of the needle 104 and a port 182 through the sidewall of the stiffener 106 (which may also be referred to as a flash port 182) can be aligned to define a passageway 186 for the passage of blood. In particular, the passageway 186 may permit a flash of blood to pass from the lumen of the needle 104 into an elongated annular lumen 188 defined by the inner surface of the catheter 102 and the outer surface of the stiffener 106. That is, blood received into the needle tip and can pass through the passageway 186 and can flow proximally through the catheter 102 to provide a visual indication to the user of a desired placement of the needle 104 (e.g., the blood may be visible through a clear, translucent, or not fully opaque catheter 102). Stated otherwise, the passageway 186 and the lumen 188 can provide a channel through which a flash of blood can pass to indicate that the distal end of the needle 104 has entered a blood vessel, such as previously described. The flash port 182 is also shown in FIGS. 11 and 20.

With reference to FIG. 2, in the pre-use configuration, each of the upper and lower actuators 155, 222 is in a fully retracted position. Stated otherwise, each of the upper and lower actuators 155, 222 is at a proximal-most or fully rearward position. Accordingly, the stiffener hub 154 is also in a fully retracted position.

In the illustrated embodiment, the upper actuator 155 does not initially engage the stiffener hub 154 when both components are in their retracted orientations. In particular, as can be seen in FIG. 2, a small space or gap is present between the engagement face 326 of the engagement protrusion 325 of the upper actuator 155 and the engagement face 381 of the stiffener hub 154. Accordingly, the upper actuator 155 is advanced forwardly a very short distance to initially engage the stiffener hub 154.

In other embodiments, the engagement faces 326, 381 of the upper actuator 155 and the stiffener hub 154 are in abutting contact in the pre-use state of the system 100, such that forward movement of the upper actuator 155 immediately achieves concurrent forward movement of the stiffener hub 154. In some instances, the presence and/or size of any initial gap between the engagement faces 326, 381 can vary from system 100 to system 100 within an acceptable tolerance range, such that no forward movement or only slight movement of the upper actuator 155 is required prior to the upper actuator 155 engaging the stiffener hub 154 for any of the systems 100 manufactured within specification.

As previously noted, the distal tip 334 of the stiffener hub 154 is complementary to the tapered proximal mouth 264 of the catheter hub core 141 and can fit snugly therein to efficiently apply deployment forces to the stiffener hub 154 in the distal direction. In the illustrated embodiment, the distal tip 334 of the stiffener hub 154 engages the proximal mouth 264 of the catheter hub core 141 while the system 100 is in the pre-use or pre-deployment state. In some instances, it can be desirable to ensure that the stiffener hub 154 engages the catheter hub core 141 in this initial state of the system 100 to ensure that the stiffener 106 and catheter 102 move substantially in unison immediately upon actuation of the stiffener hub 154. That is, the stiffener hub 154 immediately transfers force to the catheter hub core 141 such that both components move forward in unison. Such an arrangement can alleviate strain forces along the length of the catheter 106 that might otherwise arise in the absence of the stiffener hub 154 pushing the catheter hub core 141 forward.

For example, as previously discussed (with respect to other embodiments), advancement of the stiffener 106 causes the distal tip of the stiffener 106 to push forwardly on the distal tip of the catheter 102. This not only causes the distal tip of the catheter 102 to move forward, but also draws the remainder of the catheter 102 forward as well, due to stresses exerted along the length of the catheter 102. Should forward movement of the catheter hub core 141 be impeded, strain along the length of the catheter 102 can increase.

By urging the catheter hub core 141, to which the proximal end of the catheter 102 is attached, forward, the stiffener hub 154 alleviates stresses along at least a portion of the length of the catheter 102. This stress alleviation can be particularly pronounced, and particularly useful, at latter stages of the catheter deployment for certain embodiments, where increased force may need to be provided to the catheter hub core 141 to spread open the resilient arms at the distal end of the catheter connection hub 145 during coupling of the catheter hub core 141 to the catheter connection hub 145 (in manners such as previously discussed). In such instances, all or substantially all force required to couple the catheter hub core 141 to the catheter connection hub 145 can be provided directly to the catheter hub core 141 by the stiffener hub 154.

The strain relief provided to the catheter 102 by the interfacing of the stiffener hub 154 with the catheter hub core 141 can be explained in other terms. For example, by ensuring a direct coupling between the stiffener hub 154 and the catheter hub core 141 exists in the initial, pre-use state of the system 100, both the proximal ends and distal ends of the catheter 102 and the stiffener 106 move forward at the same rate. Stated otherwise, a length of the catheter 102 and a length of the stiffener 106 are each substantially constant throughout deployment, and further, the catheter 102 and the stiffener 106 move forward in unison.

In other embodiments, a space or gap may be present between the distal tip 334 of the stiffener hub 154 and the tapered proximal mouth 264 of the catheter hub core 141 when the system is in the initial or pre-use orientation, and potentially through at least some of the subsequent phases of deployment. For example, a small gap may be present due to manufacturing tolerances or the like. In certain of such instances, the stiffener hub 154 does not assist in translating the catheter hub core 141 forward unless and until sufficient strain on the catheter 102 elongates the catheter 102 into contact with the stiffener hub 154. For example, in certain of such embodiments, the catheter hub core 141 may be pulled distally by the catheter body 102 up until the catheter hub core 141 comes into contact with the resilient arms of the catheter connection hub 145. Due to the increased resistance to distal movement provided by the catheter connection hub 145, the catheter body 102 may elongate as the stiffener 106 is urged distally to the point where the stiffener hub 154 engages the proximal end 334 of the catheter hub core 141. At this point, the stiffener hub 154 can directly push on the catheter hub core 141, thereby supplementing the distal forces on the catheter hub core 141 that are also provided to the catheter hub core 141 through an indirect path-specifically, the stiffener hub 154 urges the stiffener 106 forward, which urges the distal tip of the catheter 106 forward, which pulls forward the proximal end of the catheter 102 and the catheter hub core 141 to which it is attached.

With continued reference to FIG. 2, in the illustrated embodiment, when the system 100 is in the pre-use or undeployed state, the catheter hub core 141 is spaced from the catheter connection hub 145. In particular, the catheter hub core 141 is entirely separate from the catheter connection hub 145, is not in contact therewith, and is distanced from the catheter connection hub 145 by a significant length. The catheter hub core 141 is positioned rearward of or proximal to the catheter connection hub 145. The catheter hub core 141 is positioned at an interior of the housing 142, or stated otherwise, is fully received within the cavity 202 of the housing 142. The housing may 142 may be said to encompass, encircle, or enclose the catheter hub core 141. Moreover, in the illustrated embodiment, no portion of the catheter hub core 141 is encompassed, encircled by, or enclosed by the catheter connection hub 145 when the system 100 is in the pre-deployed state.

In contrast, in the illustrated embodiment, the catheter connection hub 145 is coupled to a distal end of the housing 152 in manners such as previously disclosed. Accordingly, the catheter connection hub 145 is connected to the housing 152 via a connection interface 210. With the exception of the coupling interface 210, substantially an entire exterior surface of the catheter connection hub 145 is at an exterior of the housing 142. An interior of the catheter connection hub 145 is, however, in fluid communication with the cavity 202 of the housing 142. Further, in the illustrated embodiment, with the exception of the proximal portion of the catheter connection hub 145 that defines the connection interface 210, a substantial portion or most of the catheter connection hub 145 extends distally away from the housing 142 and is external to the housing 142.

In the initial state of the system 100, the catheter hub core 141 is free to translate within the housing 141 in manners such as previously disclosed (e.g., slide longitudinally while remaining rotationally locked). In contrast, the catheter connection hub 145 is in a selectively fixed relationship relative to the housing 142.

As further discussed below, when the system 100 is in the undeployed state, the distal end of the system 100 (e.g., the distal tips of the needle 104, the catheter 102, and stiffener 106) can be advanced through the skin of a patient, thereby establishing an insertion site of the skin (e.g., the insertion site 54 identified in FIG. 4A), and at least a distal tip of the needle 104 can further be advanced into a vessel of the patient, thereby establishing a vessel insertion site (e.g., the vessel insertion site 56 identified in FIG. 4A). In some instances, deployment of the system 100 begins after only the tip of the needle 104 has been advanced into the vessel. In other instances, the distal tip of the catheter 102—and, in further instances, the distal tip of the stiffener 106 as well—likewise enters into the lumen of the vessel through the vessel insertion site by a relatively small amount while the system 100 is in the undeployed state.

Once a suitable portion of the distal end of the system 100 is within the lumen of the vessel, as indicated by a flash of blood in manners such as previously disclosed, the system 100 can then be actuated or deployed to insert the catheter 102 into the vessel and thereafter advance the catheter 102 to a final or maximum depth within the vessel (e.g., where only the distal tip of the needle 104 was initially inserted into the vessel lumen), or to advance the catheter 102 to the final depth within the vessel (e.g., where at least the tip of the catheter 102 was also initially inserted into the vessel lumen).

In the illustrated embodiment, when the system 100 is in the retracted state, the stop 392 defined by the upper housing element 152t is positioned within a cavity 397 defined by the stiffener hub 154. The stop 392 does not restrict distal or proximal movement of the stiffener hub 154 when positioned within the cavity 397.

FIG. 14 depicts the system 100 in a subsequent operational state. In particular, the system 100 has been partially deployed or deployed an intermediate amount via the upper actuator 155. A range of partial or intermediate deployments of the system 100 are possible via the actuator 155. In the illustrated stage, the upper actuator 155 has been advanced to its maximum forward position, which has resulted in forward movement of the stiffener hub 154, and hence concurrent forward movement of the stiffener 106 and the catheter 102 through a first distance. In some embodiments, the first distance can be selected to ensure that the stiffener 106 and the catheter 102 are advanced through the lumen of the vessel to a depth sufficient to maintain the catheter 102 and the stiffener 106 within the vessel for a temporary period prior to final advancement of the catheter 102 and the stiffener 106 to the maximum deployed depth via the lower actuator 222.

Stated another way, in the operational phase depicted in FIG. 14, the upper actuator 155 has been advanced along an entirety of the open track defined by the housing 152, and thus along a predetermined distance. This forward movement of the upper actuator 155 pulls the stiffener hub 154 forward by a corresponding or roughly corresponding amount. In particular, when the upper actuator 155 is moved from its fully retracted position to its fully advanced position, as shown, the stiffener hub 154 also moves a predetermined distance, which may be the same as or substantially the same as the predetermined distance travelled by the upper actuator 155.

For reasons discussed further hereafter, the first deployment distance traveled by the stiffener hub 154 may also be referred to as a stabilization, anchoring, and/or retention distance, as advancing the catheter 102 into the vessel to this distance can help to ensure that the catheter 102 remains positioned within the vessel for at least an intermediate period. The intermediate period can begin after the initial deployment phase achieved via the upper actuator 155 has ceased and can end once actuation of the lower actuator 222 to achieve a final deployment of the system 100 begins.

In some instances, the upper actuator 155 can conveniently be advanced forwardly in a variety of ways using a single finger (e.g., the index finger) of a hand while that same hand is holding the handle 150. In the illustrated embodiment, the upper actuator 155 is at the forward end of the handle 150, which can facilitate this form of actuation.

In FIG. 14, the upper actuator 155 has been advanced fully to its distalmost, advanced position. The upper actuator 155 has pulled the stiffener hub 154 forward by the same amount, but the stiffener hub 154 remains in only a partially actuated or partially deployed state. At this operational state, the stop 392 remains within the cavity 394 of the stiffener hub 154 and thus does not inhibit movement of the stiffener hub 154. Accordingly, the additional forces that come into play when the longitudinally elongated portion of stiffener hub 154 and/or a proximal portion of the upper housing element 152t are deflected away from each other by interaction of a proximal end of the stiffener hub 154 with the the ramped surfaces of the stop 392, as discussed further below, do not have any bearing on deployment of the upper actuator 155.

FIG. 15 shows a subsequent stage in which the proximal end of the stiffener hub 154 has been advanced just past the stop 392. In the illustrated embodiment, the upper housing element 152t and the stiffener hub 154 are formed of flexible (e.g., resiliently flexible) material and also define long moment arms in the longitudinal direction, and one or both of these components can readily bend by a small amount—away from the other or away from each other—as the proximal end of the stiffener hub 154 is advanced over the ramped surfaces 393 of the stop 392 (see also FIG. 4). Due to the resiliency of the materials used, the housing element 152t and/or the elongated proximal stem of the stiffener hub 154 have resiliently returned, or snapped back, into a substantially parallel orientation relative to each other. In some instances, only a small sound or no discernable sound may be made and/or only a slight difference in pushing force may be detectable by the user when this snap back occurs so as to avoid any confusion as to when the system 100 has been fully deployed to the point that catheter hub core 141 audibly snaps into the catheter connection hub 145. Indeed, in the illustrated embodiment, all interactions between the stop 392 and the stiffener hub 154 is completed before the catheter hub core 141 is securely engaged by the flexible gripping arms of the catheter connection hub 145.

Once the stiffener hub 154 has been advanced distally to the position shown in FIG. 15, the stop 392 prevents the stiffener hub 154 from being retracted or moved proximally past the stop (unless extra and deliberate force for achieving such a retraction are exercised, such as to deliberately reset the device, as discussed below). Stated otherwise, the stop 392 can inhibit or prevent, under normal usage, the proximal retraction of the stiffener hub 154 once the distal position depicted in FIG. 15 has been reached.

In the operational stage depicted in FIG. 15, the tapered distal end 260 of the catheter hub core 141 has caused the resilient arms 277 of the catheter connection 145 to spread apart as the catheter hub core 141 has been moved distally. The distal movement has not yet been sufficient, however, for the resilient arms 277 to resiliently return to a resting state, or snap back in an inward direction, into engagement with the engagement recess 261 (described above with respect to FIGS. 7 and 8).

FIG. 16 illustrates that the stiffener hub 154 has been advanced distally by a small additional amount to permit the resilient arms 277 to engage the engagement recess 261 of the catheter hub core 141. In some instances, the increase in force required to push past the inner protrusions of the resilient arms 277 can be perceptible, relative to the forces used during all prior phases of deployment, so as to provide tactile feedback to the user regarding the status of the construction of the hub assembly 149. In some instances, the release of energy and/or click or other sound made by the resilient arms 277 as they automatically secure to the catheter hub core 141 may provide further or other feedback to the user regarding the assembly status of the hub assembly 149. A comparison between FIGS. 15 and 16 shows that in the illustrated embodiment, a small additional distal movement of the stiffener hub 154 past the stop 392 is permitted and, indeed, may be needed in some instances, to fully assemble the hub assembly 149.

In like manner, the stiffener hub 154 may be translatable by the small amount in the proximal direction, such that there is a small amount of longitudinal play in the stiffener hub 154, but substantial proximal movement of the stiffener hub 154 relative to the housing 152 is inhibited or prevented by the stop 392.

Stated otherwise, FIG. 16 depicts the system 100 in a fully deployed state, or deployment by a complete or maximum amount. The final amount of actuation has been achieved via the lower actuator 222. A range of partial or intermediate deployments of the system 100 are also possible via the lower actuator 222. However, in the illustrated stage, the lower actuator 222 has been advanced to its maximum forward position, which has resulted in forward movement of the stiffener hub 154, and hence concurrent forward movement of the stiffener 106 and the catheter 102 through an additional second distance. Accordingly, the system 100—and, specifically, the stiffener hub 154 of the system 100—has been actuated through a total distance that is the sum of the first distance mentioned above (i.e., resulting from the indirect actuation of the stiffener hub 154 via the upper actuator 155) and the second distance mentioned above (i.e., resulting from the direct actuation of the stiffener hub 154 via the lower actuator 222).

Stated otherwise, after initial actuation of the system 100 via the upper actuator 155, the lower actuator 222 can be advanced the remainder of an available forward path to finish deploying the catheter 102/stiffener 106 combination over the needle 104.

In the illustrated embodiment, no further forward movement of the upper actuator 155 occurs during direct actuation of the lower actuator 222. Stated otherwise, the upper actuator 155 may disengage from the stiffener hub 154 and remain stationary relative to the housing 152 during the further forward advancement of the stiffener hub 154.

The forward path traveled by the stiffener hub 154 can be delimited by the catheter connection hub 145. Stated otherwise, coupling of the catheter hub core 141 with the catheter connection hub 145 can terminate forward advancement of the stiffener hub 154. In particular, in the illustrated embodiment, the lower actuator 222 is urged (e.g., pressed) forward to directly advance the stiffener hub 154 forward. As previously discussed, throughout either a portion of or an entirety of this forward advancement of the stiffener hub 154, the stiffener hub 154 can engage and press on a proximal end of the catheter hub core 141, thus urging the catheter hub core 141 forwardly. The user can be provided with a tactile feedback that the catheter hub core 141 has begun engaging the resilient arms of the catheter connection hub 145 as resistance to forward movement of stiffener hub 154 can increase. Ultimately, the catheter hub core 141 is advanced distally by a sufficient amount to permit the deflected engagement arms of the catheter connection hub 145 to snap into a groove of the catheter hub core 141 and firmly hold the catheter hub core 141, as described more fully above. Because the catheter connection hub 145 is securely connected to the housing 152 and the catheter hub core 141 is securely connected to the catheter connection hub 145 at this point, the user can be prevented from advancing the stiffener hub 154 any further relative to the housing 152. This significant resistance or complete opposition to further advancement of the stiffener hub 154 relative to the housing 152 can provide further tactile feedback to the user, this time indicating that deployment is complete and the catheter assembly 149 is fully assembled.

In some embodiments, the user may also receive auditory feedback that deployment is complete. For example, the catheter connection hub 145 and/or the catheter hub core 141 may individually or in cooperation generate an auditory signal upon coupling. In the illustrated embodiment, connection of the catheter connection hub 145 to the catheter hub core 141 generates an audible “click,” indicating that coupling is complete.

In other or further instances, the forward path can be delimited by direct contact between the stiffener hub 154 and the lower housing element 152b. In any event, the stiffener hub 154 may cooperate directly or indirectly with the housing 152 to delimit forward movement of the stiffener hub 154.

As previously discussed, in some instances, the lower actuator 222 can conveniently be advanced forwardly by one hand of a user while the user holds the housing 152 with the other hand. In some instances, it may be convenient or otherwise advantageous for the lower actuator 222 to be positioned rearward of the lower housing element 152b, generally rearward of the upper actuator 155, and/or extend downward relative to the housing 152 generally. In some instances, such an arrangement can yield a compact system 100, as the lower actuator 222 does not extend significantly beyond lower profile of the lower housing element 152b. Nevertheless, the illustrated actuator 222 is sufficiently large to be readily gripped and/or readily pushed to deploy, or further deploy, the system 100. In some instances, such a significant rearward location of the lower actuator 222 can permit the handle 150 to be positioned close to the skin of the patient, which can permit shallow insertion angles.

With further reference to FIGS. 2 and 14-16, a two-actuator system 100 and two-phase deployment process, as just described can be advantageous in some instances. The first stage of actuation (e.g., an index finger flick or other advancement of the upper actuator 155) can assist in an initial capture the vessel, and the second stage of actuation can then advance the catheter 102 to its final or fully inserted position.

This may be particularly useful in deep vein placements of the catheter 102. In such placements, a practitioner may use, e.g., the nondominant hand to press against the skin above the vessel to provide tension to the region and assist in positioning the vessel and/or aligning the system 100 with the same. The other (e.g., dominant) hand can grip the system 100 (or any desired portion thereof, such as the handle 150), and advance the full system 100 forward to introduce the needle tip (and potentially the catheter tip as well, at this stage) into the vessel until a flash of blood is seen.

Removal of the non-dominant hand from the skin of the patient at this point, prior to deployment of the catheter 102 into the vessel over the needle 104, could allow sufficient shifting of the vessel and surrounding tissues, or otherwise destabilize the region and/or allow inadvertent movement of the dominant hand and the system 100 it is holding relative to the region, in a manner that the needle 104 and catheter 102 inadvertently emerge from the vessel. To prevent this, after placement of the needle tip in the vessel, it can be desirable to advance the catheter 102 (e.g., via the sheathing cannula 106) into the vessel to, e.g., at least M inch or so to prevent inadvertent removal of the catheter 102 from the vessel when the nondominant hand is removed in order to actuate the lower actuator 222 thereby. This is accomplished by advancing the upper actuator 155 forward while both hands maintain steady positioning, such as pressure on the patient with the nondominant hand and gripping of the system 100 with the dominant hand. After initial capture of the vessel in this manner and removal of the nondominant hand from the patient's skin, the nondominant hand can then be used to advance the lower actuator 222 to finish advancing the catheter 2012 into the patient to the target depth, or stated otherwise, to the fully deployed position.

Capture of the vessel in the foregoing manner may be referred to in a variety of ways. For example such vessel capture may also be referred to as stabilizing or anchoring the system 100 relative to the vessel. That is, the catheter 102 is desirably advanced to a position within the vessel that will permit retention of the catheter 102 within the vessel, despite small or inadvertent relative movements between the vessel and the system 100. Accordingly, the initial distance to which the catheter 102 is advanced over the needle within the vessel may be referred to as a capture, stabilization, anchoring, or retention distance. Such advancement of the catheter 102 is preparatory to the final deployment of the catheter 102 to its final position within the vessel, which may also be referred to as the indwelling, fully advanced, or resident position, etc.

In some instances, placement of the lower actuator 222 below the handle 150 allows the dominant hand to maintain continuous contact with the handle 150 throughout both the initial introduction of the catheter 102 into the vessel and subsequent actuation of the system 100 for further deployment the catheter 102 to a final depth within the vessel. For example, by gripping the handle 150 with the dominant hand, the fingers may wrap around the housing 152, but not extend over the pathway along which the lower actuator 222, or more generally, the stiffener hub 154, is slid. As the dominant hand grips the housing 152, with the index finger on one side and the other fingers on the other, the nondominant hand can engage the lower actuator 222 and move it forward between the thumb and fingers of the dominant hand, without disrupting placement of the thumb and fingers of the dominant hand. In other instances, the roles of the dominant and nondominant hands can be reversed.

In the illustrated embodiment, the upper actuator 155 captures or engages the stiffener hub 154 when advanced in the distal direction, but not when pulled in the proximal direction, which is depicted in FIG. 17. The stiffener hub 154 does not capture or engage the upper actuator 155 when moved in the forward direction, but the stiffener hub 154 may capture or engage the upper actuator 155 if the stiffener hub 154 is pulled in the rearward direction (if the upper actuator 155 has previously been advanced distally).

With reference again to FIG. 2, in some instances, a user may forego using the upper actuator 155 and may opt instead to use only the lower actuator 222 to deploy the catheter 102. That is, the lower actuator 222 may be moved the full deployment distance directly, or without use of the upper actuator 155, which would yield a configuration such as shown in FIG. 17. That is, by using solely the lower actuator 222, the user could move directly from the configuration of FIG. 2 to that shown in FIG. 17 (e.g., where the upper actuator 155 remains substantially unmoved relative to the housing 152). In the illustrated arrangement, due to a lack of interaction between the stiffener hub 154 and the upper actuator 155 during forward movement of only the stiffener hub 154 from the position depicted in FIG. 2, the upper actuator 155 may remain in its initial position during such a deployment. Thus, when the system 100 is in the fully deployed state, the upper actuator 155 may be positioned in a fully retracted state rather than the fully advanced state. With reference to 17, then, upon full deployment of the system 100 in this manner, the actuator 155 would be positioned in its leftmost (i.e., proximal most), rather than rightmost (i.e., distal most), orientation.

In some instances, a practitioner may opt to use such a one-stage actuation in contexts such as peripheral placements. For example, a practitioner may, in some instances, prefer to use only the lower actuator 222 to deploy the catheter 102 if vessel access is relatively straightforward. The practitioner may insert the tip of the needle 104 (and potentially the tip of the catheter) into the vessel a desired initial amount (e.g., while the system 100 is in the undeployed stated) without using the other hand for tensioning/positioning purposes, due to the relative accessibility (e.g., due to shallow position) of peripheral vessels. Once the system 100 has been inserted into the vessel to an initial depth (which may also be referred to as an introduction depth), the practitioner may then slide only the lower actuator 222 to advance the catheter 102 into the vessel to the final or indwelling depth.

Accordingly, the system 100 can be usable in two different deployment modes—i.e., in a two-phase deployment mode or a one-phase deployment mode. A user thus can select which mode to use based on preference, type of vessel being accessed, etc.

Alternatively, FIG. 17 may depict a stage of a two-actuator deployment method operation in which the upper actuator 155, after having been fully deployed distally, has been retracted proximally away from the catheter connection hub 145. In some instances, moving the upper actuator 155 in this manner can facilitate removal of the catheter connection hub 145 from the housing 152. This view also demonstrates that the upper actuator 155 does not engage the stiffener hub 154 when moved rearwardly, and thus the stiffener hub 154 remains in its forward, fully deployed position.

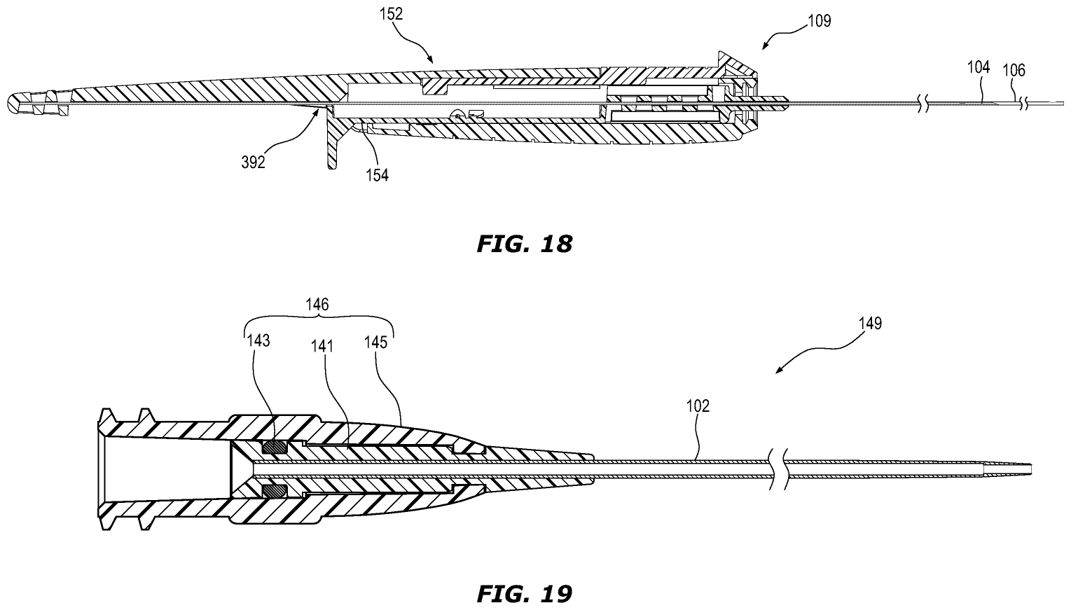

FIGS. 18 and 19 depict a later stage of operation in which the insertion assembly 109 has been removed from the catheter assembly 149 while the catheter assembly 149 is held in place relative to the patient, with the catheter 102 positioned at its indwelling position with the vessel. For example, in some instances, the user can hold the catheter assembly 149 steady or stationary relative to the patient with one hand while withdrawing the insertion assembly 109 from the catheter assembly 149 with the other.

As shown in FIG. 18, in this embodiment, the stiffener hub 154 can be permitted to move somewhat proximally relative to the housing 152, but is prevented from fully retracting to the starting position by the stop 392. In this manner, a significant length of the stiffener 106 extends distally beyond a distal tip of the needle 104 to shield the needle tip from inadvertent contact (e.g., preventing inadvertent needle sticking of the user).

As shown in FIG. 19, the catheter assembly 149, which may remain in the patient, can include the deployed catheter 102, the catheter hub core 141, a seal member 143, and the catheter connection hub 145.

With reference again to, e.g, FIG. 17, in some embodiments, the system 100 can be resettable. Stated otherwise, the non-return feature or stop 392 may be selectively overridable to permit resetting of the device. In the illustrated embodiment, resetting of the device may be achieved by bending the upper housing element 152t and/or the stiffener hub 154 away from the other (or away from each other) to move the stiffener hub 154 proximally past the stop 392 and back to the initial position of FIG. 2. In some instances, the system 100 may be used in a reset state to advance a catheter into a vessel, even after the bond between the tip of the catheter 102 and the needle 104 has been broken. For example, in some instances, the support provided by the stiffener 106 can be sufficient to assist in urging the catheter tip through the vessel wall.

Illustrative methods of using the system 100 have previously been described. Further details of certain of these or other methods will now be described.

A user of the system 100 may remove the system 100 from packaging, at which point the system 100 can be in the pre-deployment state depicted in FIG. 2. The user may prepare the skin of a patient at which an insertion site will be formed according to standard operating procedures. The user may then advance the distal end of the system 100 (such as depicted in FIG. 2) through the skin of a patient and into a vessel in manners such as previously described. Moreover, as previously described, the stiffener 106 can facilitate and/or reduce or avoid deformation of the distal tip of the catheter 102 during such insertion through the skin, as well as though the vessel wall. Once the distal end of the system 100 has been inserted into the vessel by a sufficient amount, a flash of blood will become visible indicating proper introduction into the vessel has been achieved. At this point, the tip of the needle 104 has entered the vessel, and possibly the distal tip of the catheter 102 and the distal tip of the stiffener 106 may have entered the vessel as well. To the extent the catheter 102 has entered the vessel at this point, the depth to which the catheter 102 has been inserted into the lumen of the vessel may be referred to as the introduction depth, initiation depth, preliminary depth, etc.

After viewing the flash of blood, the user may then deploy the catheter 102 over the needle 104 in any of the manners described above. For example, in some methods, the user may first advance the upper actuator 155 forward, relative to the housing 150 (which may be held substantially stationary, steady, stable, fixed, or immobile relative to the patient and/or relative to the vessel), to deploy the catheter 102 to a capture depth within the vessel, and may thereafter advance the lower actuator 222 forward, relative to the housing 150 (which, again, may be held substantially stationary, steady, stable, fixed, or immobile relative to the patient and/or relative to the vessel), to further deploy the catheter to the final indwelling depth within the vessel, and also to assemble the catheter assembly 149. In other methods, the user only utilizes the lower actuator 222 to fully deploy the catheter to the final indwelling depth, and also to assemble the catheter assembly 149. In either case, the user may be alerted that the indwelling depth has been reached via tactile feedback (e.g., difficulty advancing or inability to advance the lower actuator 222) and/or auditory feedback (e.g., clicking of the catheter assembly 149 into place).

FIG. 17 depicts the stiffener hub 154 in the fully advanced position. In many embodiments, forward movement of the stiffener hub 154 is not delimited by the housing 152 directly, but rather, is delimited by the housing 152 indirectly due to interactions and connections housing 152 and the catheter assembly 149. However, in some instances, the stiffener hub 154 may directly contact the lower housing member 152b in the illustrated operational state in an abutting fashion the delimits forward movement of the stiffener hub 154. In some instances, such contact may only occur in situations at the extreme limits of manufacturing tolerances. In other or further instances, it may be desirable to ensure that at least some space exists between any stopping surfaces of the housing 152 and the stiffener hub 154 when the stiffener hub 154 is fully advanced to ensure sufficient runway exists to permit full assembly of the catheter hub.

As previously noted, the lock 392 can retain the stiffener 106 in an advanced state over the tip of the needle 104 when the stiffener hub 154 has been advanced to the fully advanced state. By being restricted to such a forward position, the stiffener hub 154 can effectively cooperate with the lock 392 to keep the stiffener 106 positioned over or past the distal tip of the needle 104 to thereby shield the needle from inadvertent contact in manners such as previously described. Stated otherwise, in some embodiments (such as certain embodiments discussed hereafter), the stiffener hub 154 can cooperate with the housing 152 to prevent the stiffener 106 from exposing the needle tip after a deployment event. Stated otherwise, the stiffener hub 154 and the attached stiffener 106 can be restrained to a position relative to the housing 154 that maintains the stiffener 106 in a shielding orientation relative to the tip of the needle 104—e.g., in a position in which the stiffener 106 extends distally past the distal tip of the needle 104 by an amount sufficient to inhibit or prevent inadvertent contact with the needle tip.

With reference to FIG. 20, a distal and intermediate portion of an embodiment of the stiffener 106 is shown. As with other embodiments described above, the stiffener 106 can be formed of any suitable material. In some embodiments, the stiffener 106 may be of a one-piece construction. For example, in some embodiments, the stiffener 106 comprises an extruded tube of polymeric material. In certain embodiments, the stiffener 106 comprises an elongated PEEK tube. In various embodiments, the stiffener 106 may be relatively stiffer than the catheter 102.

With reference again to FIG. 2, the system 100 can include a handle 150 to which each of the needle 104, the stiffener 106, and the catheter 102 are initially coupled. The handle 150 may also be referred to herein as a housing. The needle 104 can be fixedly and permanently secured to the handle 150, the stiffener 106 can be movably and permanently coupled with the handle 150, and the catheter 102 can be movably and removably coupled with the handle 150 in manners such as previously discussed.

In the illustrated embodiment, the stiffener 106 is fixedly and permanently secured to a stiffener hub 154, which can be movably coupled with the handle 150 so as to be longitudinally advanceable relative thereto. As with other embodiments, the stiffener hub 154 can include an actuator 222 that can be engaged to advance the stiffener hub 154 relative to the handle 150. In some instances, the stiffener hub 154 may be referred to as a slider or slide. For example, in some instances, the stiffener hub 154 may be particularly well suited for actuation by the thumb of a practitioner, and may be referred to as a thumb slider or thumb slide.

With continued reference to FIG. 2, and as previously noted, the needle 104 can be fixedly and permanently secured to the handle 150. In the illustrated orientation, the stiffener hub 154 and the stiffener 106 attached thereto are in a retracted, pre-use, proximal, or undeployed position, which can be at a proximal-most position relative to the handle 150 and the needle 104. The catheter 102 is also in a retracted, pre-use, proximal, or undeployed position, which likewise can be at its proximal-most position relative to the handle 150 and the needle 104. As with other embodiments discussed herein, the catheter 102 can be fixedly secured to a hub core 141 that is movable relative to the handle 150. The hub core 141 can be advanced from a retracted position into an advanced position in which the hub core 141 coupled with a catheter connection hub 145. The catheter connection hub 145 is initially fixedly attached to the handle 150 in a removable fashion. As with other embodiments previously disclosed, after the hub core 141 is advanced into and coupled with the catheter connection hub 145 (e.g., after desired placement of the catheter 106 and full assembly of the hub core 141 and the catheter connection hub 145 to each other), the catheter connection hub 145, along with the hub core 141 and catheter 102 that have been fixedly secured thereto, can be decoupled from the handle 150 in manners such as previously described. After deployment and decoupling of the catheter 102, the handle 150 and components coupled therewith, including the stiffener 106 and the needle 104, can be removed proximally from the catheter 102 in manners such as previously discussed.

With continued reference to FIG. 2, the handle 150 can include any suitable lock or stop 392 by which the stiffener hub 154 can be maintained in the advanced position. As previously discussed, certain embodiments of the stops can permit some amount of retrograde or proximal longitudinal movement of the stiffener hub 154 after the stiffener hub 154 has been moved to its distal-most position. Although the stop 392 permits some small longitudinal movement or play of the stiffener hub 154 relative to the associated handle of that embodiment, the stop 392 nevertheless locks the stiffener hub 154 and associated stiffener 106 in a distally advanced position relative to the needle 104, which can permit shielding of the needle 104 in manners such as previously discussed. Varieties of the stops 392 such as described can serve as locks that maintain the stiffener 106 in an advanced shielding position (whether fixed or somewhat movable) relative to the needle 104. Stated otherwise, as with other embodiments, the system 100 includes a lock or non-return feature that can maintain a stiffener hub 154 in a position to maintain the stiffener 106 in a shielding orientation relative to the needle 104.

With reference again to FIG. 11, a stiffener assembly 300 can include the stiffener hub 154 fixedly secured to the stiffener 106. The stiffener hub 154 and the stiffener 106 can be secured together in any suitable manner. As shown in FIG. 11, and as described more fully below, the stiffener 106 can include both the flash port 182 and a region of weakness 310 that is positioned proximal to the flash port 182.

FIG. 20 depicts an intermediate and distal portion of the stiffener 106. The stiffener 106 can include the flash port 182 previously discussed at a distal end thereof. Proximally spaced from the flash port 182 is the region of weakness 310, which is shown and described in more detail with respect to FIGS. 21 and 22.

The region of weakness 310 can be any suitable feature that predisposes the stiffener 106 to bend thereat. For example, the region of weakness 310 can include any suitable structure, material, and/or treatment that provides the region of weakness 310 with less resistance to bending (e.g., lateral bending) than at least a portion of the stiffener 106 that extends distally from the region of weakness 310, and in further instances, may display less resistance to bending than a portion of the stiffener 106 that extends proximally from the region of weakness 310. In some embodiments, the region of weakness 310 can include one or more of one or more skives, holes, etchings, rectilinear or rounded cuts, or other material removals and/or treatments. In other or further embodiments, the region of weakness 310 can include one or more materials that differ from (e.g., have a lesser hardness than) one or more materials located in the portion of the stiffener that is distal to the region of weakness 310 (and/or in the portion of the stiffener that is proximal to the region of weakness 310).

With reference to FIG. 21, in the illustrated embodiment, the region of weakness 310 comprises a notch 312 that is cut into the sidewall of the tubular stiffener 106. The notch 312 may be cut with a curved or round blade. The notch 312 has a maximum depth N_{D} and a length N_{L}. In the illustrated embodiment, the length of the notch N_{L} corresponds with a length of the region of weakness 310. It may be desirable for the notch 312 to have a smooth or rounded surface 314, as shown in FIG. 22. Such an arrangement can eliminate or reduce the presence of stress risers at which the stiffener 106 could tear or shear.

With continued reference to FIG. 21, the stiffener 106 can be formed of a tube that has an outer diameter S_{OD} in regions other than at the notch 312. In various embodiments, it may be desirable for the depth N_{D} of the notch 312 to be no greater than a designated fraction of the outer diameter S_{OD}. In particular, it can be desirable for the notch 312 to provide a weakened area of the stiffener 106 without compromising the integrity of the stiffener 106. For example, it can be desirable for the stiffener 106 to maintain its ability to advance the catheter 102 over the needle, and thus not buckle or bend during deployment of the catheter 102. It can also or alternatively be desirable for the stiffener 106 to have sufficient tensile strength to remain intact when the stiffener 106 is retracted proximally from the catheter 102. It can also or alternatively be desirable for the stiffener 106 to be resistant to breaking or tearing at the notch 312 when the stiffener 106 is subjected to bending (e.g., repeated bending) or twisting. In various illustrative embodiments, the depth N_{D} of the notch 312 is no greater than one fourth or one third the size of the outer diameter S_{OD}.

The length N_{L} of the notch 312 may be selected to permit a gradual slope of the notch surface 314. In various embodiments, the length may be, for example, no less than 1, 1.5, or 2 times the size of the outer diameter S_{OD}.

In some embodiments, the outer diameter S_{OD} is about 0.9 mm (0.035 inches), the length N_{L} is about 1.7 mm (0.067 inches), and the depth N_{D} is within a range of from about 1.3 mm to about 0.25 mm (0.005-0.01 inches). In certain of such embodiments, a thickness of the sidewall of the stiffener tube can be about 0.05 mm (about 0.002 inches).

FIG. 23 depicts a portion of the insertion assembly 109 of the catheter deployment system 100 (e.g., of FIG. 1) after the system has been transitioned to the fully deployed or fully advanced state and after the catheter assembly 149 has been removed therefrom, such as discussed above with respect to FIGS. 18 and 19. For example, with reference to FIGS. 2 and 23 (for component identification), in the state illustrated in FIG. 23, the catheter 102 may have been advanced into and/or within a blood vessel of a patient. The stiffener hub 154 is locked into its advanced position via the lock or stop 392. In manners such as discussed previously, the handle 150 can be decoupled from the catheter connector hub 145, which has now been joined with the catheter hub core 141 and the catheter 102 by way of the deployment of the catheter 102 over the needle 104. Thus decoupled, the handle 150 can be removed proximally from the catheter assembly. In the illustrated embodiment, with the stiffener hub 154 (which is attached to the stiffener 106) being locked in fixed relation to the handle 150, the proximal removal of the handle 150 concurrently removes the stiffener hub 154 and the stiffener 106 from the catheter assembly. It is also noted that since the needle 104 is fixedly secured to the handle 150, the needle is likewise removed concurrently and in unison with the handle 150 while being in a fixed relationship relative to the stiffener 106. Stated otherwise, with the stiffener hub 154 in the locked configuration, the stiffener 106 is locked in an advanced relationship relative to the needle 104 by which the stiffener 106 can shield the needle 104 from inadvertent contact.

FIG. 24 depicts the stiffener 106 and the needle 104 in the locked and shielded orientation just described after these components have been removed from the catheter 102. In this locked state, the region of weakness 310 is distally spaced from the distal tip of the needle 104 by a distance S_{SD}. This spacing thus provides a shielding length 350 or shielding portion of the stiffener 106. As discussed elsewhere, this shielding length 350 can be sufficiently short and this resistant to buckling and bending that the shielding length 350 can provide effective protection from inadvertent contact with the distal tip of the needle. The bending region 310 provides a preferred position at which the stiffener 106 bends when the distal end encounters buckling and/or transversely directed forces.

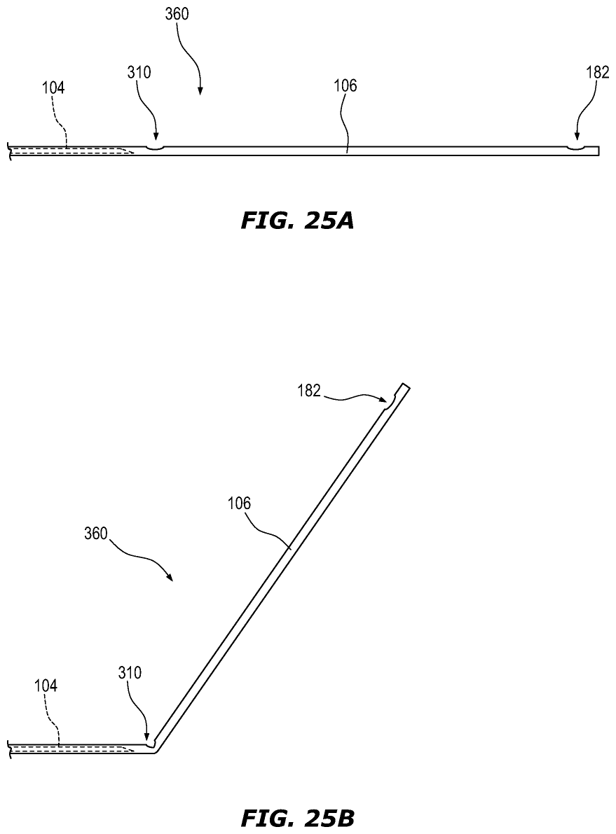

The bending may be such as shown in FIGS. 25A and 25B. The bending region 310 may, for example, define a position at which the stiffener 106 is likely to bend, and may be spaced away from the distal tip of the needle 104. The bending region 310 may be thusly spaced from the distal tip of the needle 104 by a distance that is long enough to ensure proper protection of the needle tip yet short enough to inhibit or prevent bending or buckling of the shielding length 350. In FIG. 25A, a deflectable portion 360 of the stiffener 106 that extends distally from the region of weakness 310 may be in a substantially rectilinear state, and may be linearly aligned with the portion of the stiffener 106 that extends proximally from the region of weakness 310. In FIG. 25B, the deflectable portion 360 is shown at an angled or deflected state relative to the remainder of the stiffener 106. Such a position may be the result of inadvertent contact with the deflectable portion 360 that causes such to bend relative to the needle-supported portion of the stiffener 106. In such a deflected state, the stiffener 106 can still provide effective shielding of the distal tip of the needle 104, as the shielding portion 350 that is positioned between the distal tip of the needle 104 and the bending region 310 remains in a substantially rectilinear state. The bending region 310 is sufficiently short to be resistant to bending or lateral deflection and/or buckling and is sufficiently long to prevent contact with the distal tip of the needle 104.

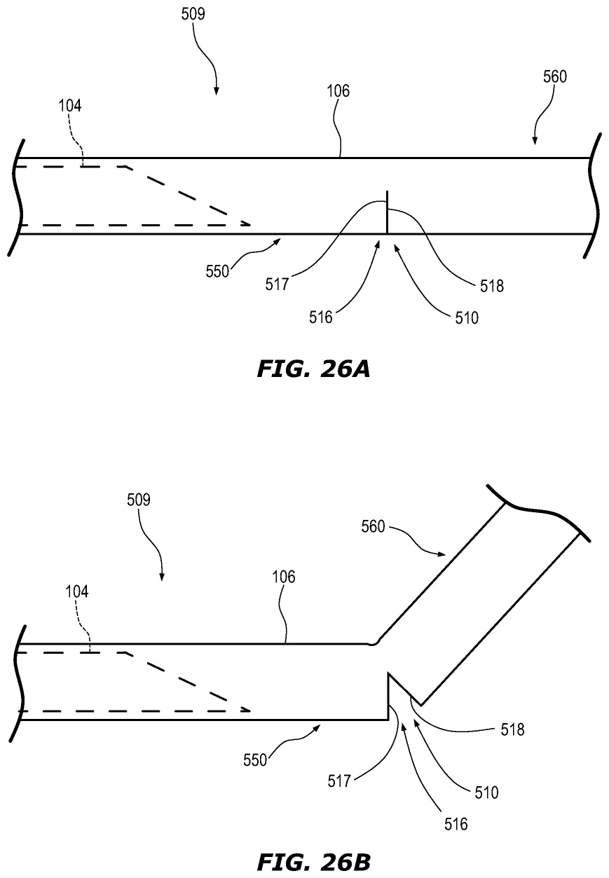

FIGS. 26A and 26B depict a further embodiment of a stiffener 106 shielding a needle 104, the stiffener 106 having a region of weakness 510 that has a different weakening feature. In particular, the region of weakness 510 is formed as a single cut 516 through the sidewall of the tubular stiffener 106. The cut 516 can yield two substantially planar surfaces 517, 518 that face one another when the stiffener 106 is in a rectilinear state. A plane that is aligned with the planar surface 517, 518 (e.g., that is parallel to or coplanar with one or more of the surfaces 517, 518) may be substantially perpendicular to a longitudinal axis of the stiffener 106. When the stiffener 106 is in a deflected state, such as shown in FIG. 26B, the planar surfaces 517, 518 can be at an angle relative to each other.

In some embodiments, an arrangement such as depicted in FIGS. 26A, 26B can be advantageous for protection the needle tip from inadvertent contact. For example, in some instances, the bending arrangement can help to ensure that the sidewall of the stiffener 106 in the shielding region 550 remains in a substantially cylindrical arrangement and/or does not flatten or bend in a manner that could bring the sidewall into close proximity or contact with the tip of the needle 104.

In some instances, however, such an arrangement may yield stress risers in the sidewall of the stiffener 106, such as, for example, at the two ends of the slice or cut 516. In certain embodiments, a tensile strength of the stiffener 106 may be significantly reduced by the presence of the cut 516.

With reference again to the stiffener 106 depicted in FIGS. 25A and 25B, in some instances, a single fixture may be used to form each of the notches 182, 312 (see FIG. 21). In some instances, the notch 310 can advantageously be devoid of stress risers and/or can maintain a relatively high tensile strength. In some instances, however, the sidewall of the tube in the shielding region 350 (FIG. 24) might flatten somewhat and/or be at an angle relative to a longitudinal axis of the needle 104/stiffener 106. This can bring the sidewall into close proximity or contact with the tip of the needle, and may render some embodiments of the stiffener 106 susceptible to needle puncture.

In various embodiments, the regions of weakness 310, 510 can be formed and/or dimensioned so as to maximize their advantages and minimize any potential drawbacks, such as herein described.

As previously discussed, in certain embodiments, the stiffener can include a region of weakness at which the stiffener is predisposed to bend. This region of weakness can be a preferred bending point for the stiffener, such that the stiffener is more likely to bend at the region of weakness than at a position at or immediately adjacent to the needle tip. The region of weakness can be distanced from the distal tip of the needle by a predetermined amount, thereby yielding a shielding length of the stiffener that remains in a substantially rectilinear state and is distal to the needle tip so as to shield the needle tip when the stiffener is bent at the region of weakness. The shielding portion of the stiffener that is positioned longitudinally between the needle tip and the region of weakness can be sufficiently long to shield the needle tip, such as by preventing inadvertent contact therewith and/or by preventing the needle tip from puncturing through the sidewall of the folded-over portion of the stiffener, while also being sufficiently short to inhibit bending or buckling thereat. For example, this shielding portion of the stiffener may be sufficiently short to have a relatively high resistance to lateral bending and a relatively high resistance to longitudinal buckling. With the stiffener being predisposed to bend at a specific position, a distal end of the stiffener may strategically or sacrificially bend laterally in the event of inadvertent contact therewith. With the overall length of the stiffener that extends past the distal tip of the needle thus reduced, the shielding length of stiffener that remains unbent—that is, the shielding length of the stiffener that extends between the region of weakness and the distal tip of the needle—can provide robust and reliable shielding or protection to the distal tip of the needle.

### Examples

Following are illustrative examples of devices, systems, and methods consistent with the present disclosure, including the written description and/or drawings.

Example 1. An apparatus comprising:
a needle that comprises a distal tip configured to be inserted into a vessel of a patient, the needle defining an outer surface;
a catheter that comprises a distal end and an inner surface that defines a lumen through which the needle extends; and
a longitudinally extending stiffener positioned between the outer surface of the needle and the inner surface of the catheter, the stiffener being configured to move to a distally advanced position relative to the needle to deploy the catheter, the stiffener further being configured to be removed from the catheter along with the needle while in the distally advanced position, the stiffener comprising a region of weakness at which the stiffener is predisposed to bend, wherein the region of weakness is closer to the distal tip of the needle than to the distal tip of the stiffener when the stiffener is in the distally advanced position.

As an illustrative example consistent with the foregoing, FIG. 23 depicts an instance in which the region of weakness 310 is closer to the distal tip of the needle 104 than it is to the distal tip of the stiffener 106.

Example 2. An apparatus comprising:
a needle that comprises a distal tip configured to be inserted into a vessel of a patient, the needle defining an outer surface;
a catheter that comprises a distal end and an inner surface that defines a lumen through which the needle extends; and
a longitudinally extending stiffener positioned between the outer surface of the needle and the inner surface of the catheter, the stiffener being configured to move to a distally advanced position relative to the needle to deploy the catheter, the stiffener further being configured to be removed from the catheter along with the needle while locked in the distally advanced position, the stiffener comprising a region of weakness at which the stiffener is predisposed to bend, wherein the stiffener is more prone to bend laterally at the region of weakness than it is to bend laterally at a length of the stiffener that is at and adjacent to the distal tip of the needle when the stiffener is locked in the distally advanced position, wherein the region of weakness is positioned distal to said length of the stiffener.

As an illustrative example consistent with the foregoing, FIG. 23 depicts an illustrative embodiment of a shielding length 350 that is at and adjacent to the distal tip of the needle 104. The region of weakness 310 is positioned distal to the shielding length 350.

Example 3. An apparatus comprising:
a needle that comprises a distal tip configured to be inserted into a vessel of a patient, the needle defining an outer surface;
a catheter that comprises a distal end and an inner surface that defines a lumen through which the needle extends; and
a longitudinally extending stiffener positioned between the outer surface of the needle and the inner surface of the catheter, the stiffener being configured to move distally relative to the needle to deploy the catheter, the stiffener comprising a region of weakness at which the stiffener is predisposed to bend, wherein the region of weakness is positioned proximally from a distal tip of the stiffener by a distance that is no less than 10 times greater than a maximum diameter of the stiffener.

As an illustrative example consistent with the foregoing, FIGS. 20 and 23 depict an illustrative embodiment of a stiffener 106 that includes a region of weakness 310. The region of weakness 310 is positioned proximally from a distal tip of the stiffener 106 by a substantial distance. In various embodiments, this distance is no less than 5, 6, 7, 8, 9, 10, 15, or 20 times greater than a maximum diameter of the stiffener 106.

Example 4. The apparatus of any preceding Example, wherein the stiffener further comprises a flash port through which blood can flow.

Example 5. An apparatus comprising:
a needle that comprises a distal tip configured to be inserted into a vessel of a patient, the needle defining an outer surface;
a catheter that comprises a distal end and an inner surface that defines a lumen through which the needle extends; and
a longitudinally extending stiffener positioned between the outer surface of the needle and the inner surface of the catheter, the stiffener being configured to move distally relative to the needle to deploy the catheter, the stiffener comprising a flash port through which blood can flow and a region of weakness at which the stiffener is predisposed to bend, wherein the region of weakness is separate from and positioned proximal to the flash port.

As an illustrative example consistent with the foregoing, FIG. 20 depicts an illustrative embodiment of a stiffener 106 that includes a flash port 182 and a region of weakness 310. The region of weakness 310 is separate from and positioned proximal to the flash port 182.

Example 6. The apparatus of any preceding Example, wherein the stiffener comprises an elongated tube.

Example 7. The apparatus of Example 6, wherein the region of weakness comprises a notch in the sidewall of the tube.

Example 8. The apparatus of Example 7, wherein the notch extends inwardly relative to an outer profile of the tube to a maximum depth and the outer profile of the tube defines an outer diameter, and wherein the maximum depth is no greater than one third the size of the outer diameter.

Example 9. The apparatus of Example 7 or Example 8, wherein the notch is devoid of angled surfaces.

Example 10. The apparatus of any one of Examples 7 to 9, wherein the notch is formed by skiving the stiffener with a curved blade.

Example 11. The apparatus of any one of Examples 7 to 10, wherein an entirety of the notch defines a rounded profile.

Example 12. The apparatus of any one of Examples 7 to 11, wherein an entirety of the notch defines a semi-circular profile.

Example 13. The apparatus of Example 6, wherein the region of weakness comprises a substantially planar cut in a sidewall of the tube.

Example 14. The apparatus of any preceding Example, wherein the region of weakness is positioned distally relative to the distal tip of the needle when the stiffener is fully advanced relative to the needle.

Example 15. The apparatus of Example 14, wherein the region of weakness remains at a position that is distal to the distal tip of the needle when the stiffener and the needle are removed proximally from the catheter.

Example 16. The apparatus of Example 15, wherein the stiffener and the needle are configured to be in a longitudinally locked configuration relative to each other when they are removed proximally from the catheter.

Example 17. The apparatus of any preceding Example, wherein the region of weakness is reinforced by the needle when the stiffener is in an initial position prior to deployment of the catheter, and wherein the region of weakness is configured to be unsupported by the needle when the stiffener is in a (or the) distally deployed position.

Example 18. The apparatus of any preceding Example, wherein the stiffener and the needle are in a coupled configuration when both are removed from the catheter.

Example 19. The apparatus of any preceding Example, wherein the stiffener is configured to lock into a fixed longitudinal position relative to the needle when the stiffener is fully distally advanced.

Example 20. The apparatus of any preceding Example, wherein a rigid columnar portion of the stiffener extends between the region of weakness and a portion of the stiffener that encompasses the needle when the stiffener is fully distally advanced relative to the needle.

Example 21. The apparatus of Example 20, wherein the rigid columnar portion is more resistant to lateral bending than is that portion of the stiffener that is positioned distal to the region of weakness.

Example 22. The apparatus of Example 20 or Example 21, wherein the rigid columnar portion is substantially shorter than that portion of the stiffener that is positioned distal to the region of weakness.

Example 23. The apparatus of any one of Example 20 to Example 22, wherein the rigid columnar portion is substantially less prone to lateral bending than is the region of weakness.

Example 24. The apparatus of any one of Example 20 to Example 23, wherein the rigid columnar portion is resistant to bending laterally in a manner that would cause a sidewall thereof to extend laterally over the distal tip of the needle.

Example 25. The apparatus of any one of Example 20 to Example 24, wherein a portion of the stiffener that extends proximally from the rigid columnar portion of the stiffener is reinforced by the needle when the stiffener is fully distally advanced relative to the needle.

Example 26. The apparatus of any preceding Example, wherein creation of the region of weakness reduces a bending stiffness of the stiffener such that the stiffener is predisposed to bend laterally at the region of weakness without reducing a tensile strength of the stiffener by an amount greater than 20, 25, 30, 35, 40, 45, or 50 percent.

Example 27. The apparatus of any preceding Example, wherein creation of the region of weakness reduces a bending stiffness of the stiffener, such that the stiffener is predisposed to bend laterally at the region of weakness, without reducing a tensile strength of the stiffener by an amount greater than 5, 10, or 15 percent.

Example 28. The apparatus of any preceding Example, wherein the stiffener comprises a polymeric tube.

Example 29. The apparatus of any preceding Example, wherein the stiffener comprises a PEEK tube.

Example 30. The apparatus of any preceding Example, wherein each of the needle and the stiffener is coupled with a handle.

Example 31. The apparatus of Example 30, wherein the needle is fixedly secured to the handle and the stiffener is moveably coupled with the handle.

Example 32. The apparatus of Example 31, wherein the stiffener is configured to move over the needle from a retracted position distally to an advanced position.

Example 33. The apparatus of Example 32, wherein the stiffener is coupled with an actuator via which the stiffener is movable from the retracted position to the advanced position.

Example 34. The apparatus of Example 32 or Example 33, wherein the stiffener is configured to be locked in the advanced position once moved thereto.

Example 35. The apparatus of any one of Examples 30 to 34, wherein the handle, the needle, and the stiffener are configured to be removed from the catheter in unison after placement of the catheter within the vessel of the patient.

Example 36. The apparatus of Example 1, wherein the stiffener comprises a distal end that engages the catheter when the catheter and the stiffener are inserted distally into the vessel of the patient over the needle.

Example 37. The apparatus of Example 36, wherein the stiffener is configured to disengage from the catheter and translate proximally relative thereto to leave the catheter positioned within the vessel of the patient as the stiffener is withdrawn from the catheter in a proximal direction.

Example 38. The apparatus of any one of Example 1 to Example 37, wherein the catheter further comprises a catching region of reduced diameter that extends inwardly at the distal end of the catheter.

Example 39. The apparatus of Example 38, wherein the distal end of the stiffener engages the catching region of the catheter when the catheter and the stiffener are inserted distally into the vessel of the patient over the needle.

Example 40. The apparatus of Example 39, wherein the catching region of the catheter comprises an inwardly directed surface in contact with a distal tip of the tube.

Example 41. The apparatus of Example 39, wherein the catching region defines a distally directed recess sized to receive at least a portion of the distal tip of the tube.

Example 42. The apparatus of any preceding Example, wherein the stiffener comprises a tube and the region of weakness comprises a notch in the tube, wherein the needle comprises a bevel having a distal end and a proximal end, and wherein the proximal end of the bevel and the notch are positioned at the same lateral side of the tube when the stiffener is in an advanced position relative to the needle.

Example 43. A method comprising:
advancing a catheter distally past a distal tip of a needle via a stiffener that comprises a region of weakness at which the stiffener is predisposed to ben;
removing the needle and the stiffener from the catheter; and
bending the stiffener at the region of weakness to shield the distal tip of the catheter.

Example 44. The method of Example 43, wherein the stiffener is positioned at an interior of the catheter during said advancing.

Example 45. The method of Example 43 or Example 44, wherein at least a portion of the needle is positioned at an interior of the stiffener during said advancing.

Example 46. The method of any one of Examples 43 to 45, wherein at least a portion of the needle is positioned within at interior of the needle during said advancing.

Example 47. The method of any one of Examples 43 to 46, wherein the catheter comprises a catching region of reduced diameter that extends inwardly at the distal end of the catheter, and wherein said advancing comprises pressing on the catching region of the catheter via the stiffener.

Example 48. The method of Example 47, further comprising inserting the catheter and the stiffener distally into a vessel of the patient over the needle while the distal end of the stiffener engages the catching region of the catheter.

Example 49. The method of Example 47 or Example 48, wherein the catching region of the catheter comprises an inwardly directed surface in contact with a distal tip of the stiffener.

Example 50. The method of any of Examples 43 to 49, wherein the stiffener comprises a tube and the region of weakness comprises a notch in the tube, wherein the needle comprises a bevel having a distal end and a proximal end, and wherein the proximal end of the bevel and the notch are positioned at the same lateral side of the tube when the stiffener is in an advanced position relative to the needle.

Example 51. The method of any of Examples 43 to 50, further comprising locking the stiffener relative to the needle.

Example 52. The method of claim 51, wherein said locking takes place prior to said removing the needle and the stiffener from the catheter.

Example 53. The method of any of Examples 43 to 52, wherein the stiffener is predisposed to bend at the region of weakness.

Example 54. The method of any of Examples 43 to 53, wherein the stiffener comprises a tube, and wherein the region of weakness comprises a cut or a notch in a sidewall of the tube.

Example 55. The method of any of Examples 43 to 54, wherein an insertion assembly comprises a handle, the needle, and the stiffener coupled together.

Example 56. The method of Example 55, wherein the needle is fixedly secured to a handle.

Example 57. The method of Example 55 or Example 56, wherein the stiffener is selectively movable relative to the handle and needle when in an unlocked state.

Example 58. The method of any of Examples 55 to 57, wherein movement of the stiffener relative to the handle and the needle is restricted when the stiffener is in a locked state.

Example 59. The method of Example 58, wherein movement of the stiffener relative to the handle and the needle is prevented in when the stiffener is in the locked state.

Example 60. The method of any of Examples 55 to 59, wherein a catheter assembly comprises the catheter, and wherein said advancing the catheter comprises moving at least a portion of the catheter assembly relative to the handle.

Example 61. The method of Example 60, wherein a catheter connection hub portion of the catheter assembly is secured to the handle during said advancing the catheter.

Example 62. The method of Example 61, wherein a catheter hub core is fixedly secured to the catheter and is advanced distally in unison with the catheter during said advancing the catheter.

Example 63. The method of Example 62, further comprising connecting the catheter hub core to the catheter connection hub prior to said bending the stiffener.

Example 64. The method of any of Examples 55 to 63, wherein said removing the needle and the stiffener from the catheter comprises withdrawing the handle, the needle, and the stiffener in unison in a proximal direction relative to the catheter.

Example 65. The method of any of Examples 43 to 64, wherein the region of weakness is closer to the distal tip of the needle than to the distal tip of the stiffener during said bending the stiffener.

Example 66. The method of any of Examples 43 to 65, further comprising locking the stiffener in a distally advanced position after said advancing, wherein the stiffener is more prone to bend laterally at the region of weakness than it is to bend laterally at a length of the stiffener that is at and adjacent to the distal tip of the needle when the stiffener is locked in the distally advanced position.

Example 67. The method of Example 66, wherein the region of weakness is positioned distal to said length of the stiffener during said bending the stiffener.

Example 68. The method of any of Examples 43 to 67, wherein the region of weakness is positioned proximally from a distal tip of the stiffener by a distance that is no less than 10 times greater than a maximum diameter of the stiffener.

Example 69. The method of any of Examples 43 to 69, wherein the stiffener further comprises a flash port through which blood can flow, and wherein the region of weakness is separate from and positioned proximal to the flash port.

It will be understood by those having skill in the art that changes may be made to the details of the above-described embodiments without departing from the underlying principles presented herein. For example, any suitable combination of various embodiments, or the features thereof, is contemplated.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

As used in this specification and the appended claims, the singular forms “a,” “an” and “the” include plural referents unless the context clearly dictates otherwise. Thus, for example, reference to “a layer” can include a plurality of such layers.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the component structures, steps, or the like specifically listed in conjunction with such terms, as well as that which is in accordance with U.S. patent law.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in any suitable manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly-complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Moreover, for references to approximations (which are made throughout this specification), such as by use of the terms “about” or “approximately,” or other terms, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially perpendicular” is recited with respect to a feature, it is understood that in further embodiments, the feature can have a precisely perpendicular orientation.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

References throughout this specification to “an example,” if any, mean that a particular feature, structure, or characteristic described in connection with the example is included in at least one embodiment. Thus, appearances of the phrases “in an example” in various places throughout this specification are not necessarily all referring to the same embodiment.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of claims [x] through the claim that immediately precedes this one” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claims 1, 2, or 3, with these separate dependencies yielding three distinct embodiments; claim 5 can depend from any one of claims 1, 2, 3, or 4, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed in accordance with 35 U.S.C. § 112(f). Elements not presented in requisite means-plus-function format are not intended to be construed in accordance with 35 U.S.C. § 112(f). Embodiments of the invention in which an exclusive property or privilege is claimed are defined as follows.

1. An apparatus comprising:
a needle that comprises a distal tip configured to be inserted into a vessel of a patient, the needle defining an outer surface;
a catheter that comprises a distal end and an inner surface that defines a lumen through which the needle extends; and
a longitudinally extending stiffener positioned between the outer surface of the needle and the inner surface of the catheter, the stiffener being configured to move to a distally advanced position relative to the needle to deploy the catheter, the stiffener further being configured to be removed from the catheter along with the needle while in the distally advanced position, the stiffener comprising a region of weakness at which the stiffener is predisposed to bend, wherein the region of weakness is closer to the distal tip of the needle than to the distal tip of the stiffener when the stiffener is in the distally advanced position.

2. The apparatus of claim 1, wherein the stiffener is configured to be locked relative to the needle in the distally advanced position and removed from the catheter while locked, wherein the stiffener is more prone to bend laterally at the region of weakness than it is to bend laterally at a length of the stiffener that is at and adjacent to the distal tip of the needle when the stiffener is locked in the distally advanced position, wherein the region of weakness is positioned distal to said length of the stiffener.

3. The apparatus of claim 1, wherein the stiffener is configured to move distally relative to the needle to deploy the catheter, and wherein the region of weakness is positioned proximally from a distal tip of the stiffener by a distance that is no less than 10 times greater than a maximum diameter of the stiffener.

4. The apparatus of claim 1, wherein the stiffener further comprises a flash port through which blood can flow.

5. The apparatus of claim 4, wherein the region of weakness is separate from and positioned proximal to the flash port.

6. The apparatus of claim 1, wherein the stiffener comprises an elongated tube.

7. The apparatus of claim 6, wherein the region of weakness comprises a notch in the sidewall of the tube.

8. The apparatus of claim 7, wherein the notch extends inwardly relative to an outer profile of the tube to a maximum depth and the outer profile of the tube defines an outer diameter, and wherein the maximum depth is no greater than one third the size of the outer diameter.

9. The apparatus of claim 7, wherein the notch is devoid of angled surfaces.

10. The apparatus of claim 7, wherein the notch is formed by skiving the stiffener with a curved blade.

11. The apparatus of claim 7, wherein an entirety of the notch defines a rounded profile.

12. The apparatus of claim 7, wherein an entirety of the notch defines a semi-circular profile.

13. The apparatus of 6, wherein the region of weakness comprises a substantially planar cut in a sidewall of the tube.

14. The apparatus of any preceding claim, wherein the region of weakness is positioned distally relative to the distal tip of the needle when the stiffener is fully advanced relative to the needle.

15. The apparatus of claim 14, wherein the region of weakness remains at a position that is distal to the distal tip of the needle when the stiffener and the needle are removed proximally from the catheter.

16. The apparatus of claim 15, wherein the stiffener and the needle are configured to be in a longitudinally locked configuration relative to each other when they are removed proximally from the catheter.

17. The apparatus of claim 1, wherein the region of weakness is reinforced by the needle when the stiffener is in an initial position prior to deployment of the catheter, and wherein the region of weakness is configured to be unsupported by the needle when the stiffener is in the distally advanced position.

18. The apparatus of claim 1, wherein the stiffener and the needle are in a coupled configuration when both are removed from the catheter.

19. The apparatus of claim 1, wherein the stiffener is configured to lock into a fixed longitudinal position relative to the needle when the stiffener is fully distally advanced.

20. The apparatus of claim 1, wherein a rigid columnar portion of the stiffener extends between the region of weakness and a portion of the stiffener that encompasses the needle when the stiffener is fully distally advanced relative to the needle.

21-42. (canceled)
